(19) United States
(12) Patent Application Publication
(10) Pub. No.: US 2010/0009335 A1
(43) Pub. Date: Jan. 14, 2010

(54) TEMPERATURE-REGULATED CULTURE PLATES

(76) Inventors: Victor Joseph, Fremont, CA (US); Amjad Huda, Fremont, CA (US); Joseph F. Rogers, Sunnyvale, CA (US)

Correspondence Address: MORRISON & FOERSTER LLP, 755 PAGE MILL RD, PALO ALTO, CA, 94304-1018, US

(21) Appl. No.: 12/278,570

(22) PCT Filed: Feb. 7, 2007
(86) PCT No.: PCT/US07/03401

§ 371 Date: Apr. 28, 2009

Related U.S. Application Data
(60) Provisional application No. 60/771,349, filed on Feb. 7, 2006.
(60) Provisional application No. 60/776,547, filed on Feb. 24, 2006.
(60) Provisional application No. 60/778,860, filed on Mar. 3, 2006.
(60) Provisional application No. 60/786,520, filed on Mar. 27, 2006.
(60) Provisional application No. 60/799,213, filed on May 9, 2006.
(60) Provisional application No. 60/857,543, filed on Nov. 7, 2006.

(51) Int. Cl. C12Q 3/00; C12M 1/36

(52) U.S. Cl. 435/3; 435/286.1

(57) ABSTRACT

Described herein are environmentally isolated tissue culture devices that may be used for cell culture, as well as systems including these devices and methods for using them. These devices may include control features for regulating the micro-environment within a well or wells of the device. For example, on-board features may regulate the temperature, humidity, pH, media level, media composition, CO₂/O₂/N₂ levels, drug concentration, cell density, byproduct (or product) production, and mixing of materials within the chamber. Material may be added to or withdrawn from the wells of the device without opening the device. Also described herein are controllers for analyzing and controlling the micro- environment within the well. Thus, the plates described herein may be used without requiring a separate incubator, allowing cells to be analyzed (e.g., imaged) continuously, allowing real-time reactions while monitoring under a microscope for hours, days or even weeks.

### CROSS-REFERENCE TO RELATED APPLICATIONS

This application claims priority to US Provisional Patent Applications: Serial No. U.S. 60/771,349, titled “Multi-Well Plates” (filed Feb. 7, 2006); U.S. 60/776,547, titled “Multi-Well Plates II” (filed Feb. 24, 2006); U.S. 60/778,860, titled “Multi-Well Plates III” (filed Mar. 3, 2006); U.S. 60/786,520, titled “MULTI-WELL PLATES IV (filed Mar. 27, 2006); U.S. 60/799,213, titled “MULTI-WELL PLATES V” (filed May 9, 2006), and U.S. 60/857,543, titled “MULTI-WELL PLATES VI” (filed Nov. 7, 2006). Each of these patent applications is herein incorporates this by reference in its entirety.

### FIELD

The present invention relates to a tissue culture device, and more particularly, concerns a multi-well tissue culture assembly for in vitro cultivation of cells in growth media, with features to isolate different chambers of the assembly, control the temperature of the growth media, maintains visibility for imaging and for controlling or regulation of growth conditions, including controlled regulation and monitoring of media gasses (e.g., O₂, CO₂, N₂, etc.), pH, cell density, product concentration, temperature, agitation, and the like. Methods of manufacturing the assembly are also described.

### BACKGROUND OF THE INVENTION

Tissue culture assemblies, frequently referred to as tissue culture plates, are commonly used for in vitro cultivation of cells particularly for experimental purposes. Multi-well tissue culture plates have been used for these purposes, and include six, twelve, twenty-four, forty-eight, ninety-six, etc. wells. Such multi-well tissue culture plates are convenient for the investigator in order to conduct tests for the separation of individual cell cultures while maintaining the cultures in close proximity (all in one plate with a single lid) for performance of related tests or assays on all the cultures.

Presently known and available tissue culture plates have been designed to operate in conjunction with an incubator which can help maintain the proper temperature, pH and gas balance (which may be related to pH) of the tissue culture media, therefore allowing optimal conditions for cell and tissue growth and maintenance. However, currently available tissue culture plates (including lids) for multi-well tissue culture have many limitations. First, media may be added or removed only be removing the lid and disturbing the potentially sensitive growth environment, and potentially exposing the chamber to contamination. Furthermore, the temperature, pH and other features of each well are typically controlled by controlling the entire incubator. Finally, visualization or analysis of cells cultured in traditional tissue-culture dishes typically requires removal of the cell culture dishes from the optimized or controlled environment of the incubator, potentially exposing the cells or tissue to stress.

Multi-well tissue culture assemblies are exemplified in U.S. Pat. Nos. 4,349,632; 4,038,149; 4,012,288; 4,010,078; 3,597,326 and 3,107,204. Another culture vessel is exemplified in U.S. Pat. No. 4,358,908. However, none of the inventions described in the above-listed patents overcomes the problems resulting from having to open the tissue culture assembly to monitor and/or regulate tissue culture media, or the additional problems associated with having to use a tissue culture incubator.

Furthermore, monitoring of morphological, physiological, and metabolic changes in cell cultures (e.g., explant or tissue cultures) often requires that the tissues be kept alive for extended periods of time. Long-term monitoring is preferable. Microscopic and/or metabolic examinations for up to 24 hours or more must be done under ideal conditions of nutrient, gas and temperature control.

Although there have been attempts to create sample holders that may be interfaced with heaters or sources of media and/or gasses (for example, US 2006/0216211 to Liebel et al.), these designs are not configured to allow visualization through the heaters, or typically use with inverted microscopes. Furthermore, these devices do not allow for closed-environment handling (e.g., sensing, stirring, media change, and temperature regulation) all on-dish, without requiring disruption or interruption (and potentially contamination) or alteration of the micro-environment of the well.

Described herein are tissue culture devices (e.g., multi-well tissue culture plates or slides) that may address some of the problems described above. For example, the smart slides described herein may allow long-term cell culture by controlling the culture conditions within the chambers of the tissue culture devices, while simultaneously allowing imaging. Thus, the devices and systems described herein may reduce or eliminate the process of going back and forth from an incubator to a microscope. Molecular and in vivo imaging approaches (including long-term or time-lapse imaging) may help the physiological role the cells play in their microenvironment.

The systems described herein provide a long-term imaging enabling platform that can keep any cells (particularly adherent cells) alive for long durations in the chambers of the plates described. These devices can therefore create user defined microenvironment in each one of the wells, while the cells or tissue are monitored (e.g., on an inverted microscope) without having to take the plate in and out of an incubator. Molecular and in vivo imaging approaches may be used on the cells within the plates, providing a valuable tool for helping to determine physiological roles of many different cells and tissues act in a viable culture environment.

### BRIEF SUMMARY OF THE INVENTION

Described herein are plates that may be used for cell culture including “smart” control features that may be sensor controlled and/or user controlled. These plates may be multi-well plates. Plates may control the microenvironment within individual or all of the wells on a plate. For example, on-board features may regulate the temperature, humidity, pH, media level, media composition, CO₂/O₂/N₂ levels, drug concentration, cell density, byproduct (or product) production, and mixing of media within the chamber. Thus, the plates described herein may be used without requiring a separate incubator, allowing cells to be analyzed (e.g., imaged) continuously, allowing real-time reactions while monitoring under a microscope for hours, days or even weeks.

Applicants have recognized that a tissue culture device, particularly a multi-well tissue culture device, may include on-board control of any or all of the features (e.g., temperature, media characteristics (e.g., gas concentrations, pH, etc.). The devices, methods and systems described herein may be used to control any of the aspects of cell culture and maintenance within a chamber, while allowing the visualization of the cells within the chamber. Also described herein are systems including the multi-well tissue culture devices as well as various additional sensors, controllers, microscopes, and imaging devices.

The devices (e.g., tissue culture plates) described herein may be referred to as smart slides, or smart plates, because they include one or more control features for monitor and/or regulating the growth media provided to cells cultured within one or more chambers of the device. Although the devices described herein are primarily shown and exemplified as tissue culture plates, they may be used for any appropriate use in which it would be desirable to control the environment of one or more wells or chambers of a plate (e.g., controlling the temperature, pH, fluid content delivered to or taken from the chamber, gas applied to or removed from the chamber, stirring the chamber, cell counting, production of protein, antibody, etc.). For example, the devices described herein may be used for controlling (or monitoring) the mixing of reactants (e.g., chemical reactants), for cell growth (e.g., bacterial cell growth) or fermentation, for immunoassays (e.g., automating fixation, washing, labeling and/or imaging), or the like.

The plates devices described herein may be useful as part of one or more methods for drug interaction/response/development, cancer research, stem cell research, cell and vascular biology research, cell morphology analysis, enzyme kinetics studies, developmental biology research, drug development, signal transduction analysis, apoptosis studies, tuberculosis testing, calcium assays, toxicology assays (panels), membrane dynamics analysis, neuronal outgrowth studies, growth factor studies, mitosis, and AIDS/HIV research or testing. Examples of some of these methods are described herein. The multi-well plates described herein may be advantageously incorporated into any method involving the use of passive multi-well plates, eliminating the need for a separate incubator, stir plate, and separate monitors, and allowing continuous monitoring.

The tissue-culture devices described herein may include any or all of the features described herein, including but not limited to: control of well temperature (maintain cells at physiologic temperatures for prolonged cell life and extended experimentation), regulation of gasses such as CO₂/O₂/N₂ (e.g., helping to maintain proper pH throughout experiment), regulation of media delivery (allows for feeding, washing and reagent delivery while imaging), drug delivery (e.g., controlled application of a drug or compound), thin (e.g., cover slip-thickness) well bottoms (permits imaging using inverted microscopes), optically clear well bottoms (permits use of standard light and fluorescence microscopy techniques with no condensation on top cover), disposable (the entire tissue-culture device may be used for a single-use, e.g., disposable, or may be configured to be sterilized and re-used), cell counting, monitoring of production of reactant or cellular byproduct (e.g., proteins, antibodies, etc.), and multiple wells (provides flexibility in experimental design). An objective heater may be included to allow the device to be used with oil immersion lenses.

Software for controlling or integrating with the devices described herein may be used to permit user control of any of the aspects described herein (e.g., programmable temperatures, flow rates, wash cycles & temperature cycles).

The plates described herein may include a lid that can be kept at a temperature slightly above the temperature of the bottom heater to eliminate condensation on lid. The lid may also include some or all of the controllable features (e.g., heating, fluid addition, gas perfusion, measurement features, etc.). Including features on the lid may allow the lid to be re-used, while disposing of the lower region housing the chambers.

In some variations, each of the wells (e.g., six 35 mm ID wells) is totally isolated from each other. Multiple ports may be provided into and out of each well. The ports may be regulated (e.g., by a valve) manually or automatically. The ports may also include a filter (or filters) preventing contamination or removing particulates. One or more gas vents may also be included (and may also be regulated by valves, such as an overflow valve, or an overpressure valve). Ports may also include splits (e.g., “Y”s) for addition of material (e.g. by injection) into the port, and ports may be connected to any appropriate tubing, or the like. Biological isolation of each well may be maintained by the use of seals (e.g., O-ring seals, crush seals, etc.) between interlocking components of each well (e.g., between the lid and the well, between the bottom of the well and the walls, etc.). In addition, each well may include one or more sampling ports configured as septum ports through which a sample can be taken without breaking the seal. A septum port may comprise a material (e.g., an elastomeric material) through which a needle or other sampling device may be inserted (e.g., by piercing the material) and removed without breaking the isolation of the chamber.

The slide may be part of a system that includes a sterile, disposable cell culture slide that has a specially coated cover slip bottom to permit thermoregulation of the slide, as described further below. The system may include a controller that connects to a computer and, via software and/or hardware interfaces (e.g., “SmartWare software”), manages the internal environment (e.g., temperature of the slide, CO₂, nutrient flow, etc.) at programmed levels. The system may also monitor the environment of the slide, and record conditions within the slide. The system may further include a monitoring system such as a microscope, camera (including video), etc. Alarms may also be included to warn (visually or by sounding an alarm) that a condition (e.g., temperature, pH, fluid level, pressure, O₂/CO₂/N₂ levels, etc.) have exceed or fallen below a threshold range.

Any appropriate imaging platforms may be used. The plates described herein are adaptable to individual system needs. For example, the smart slides described herein may be used with an inverted microscope with an oil immersion objective or a water immersion lens, an upright microscope, etc. The systems described herein may include an objective heater for controlling the temperature of the objective lens (preventing disruption of the temperature control of the slide when used with an oil immersion or water immersion lens).

The slides and systems described herein make it possible to investigate, in real-time, biological questions that are either temperature and/or time dependent by eliminating the need for a separate incubator (and stir plate, etc.), approximating an in-vivo environment under the microscope.

In some variations, the systems described herein include a fluid controller including fluid flow components and supply and return bottles. The fluid controller may include a pump (or pumps), a valve (or valves) for controlling the application of material through the ports, filters, and connectors to liquid (e.g., media, etc.) or gas (e.g., O₂/CO₂/N₂ gas supplies or mixes) to manage CO₂ levels, supply fresh nutrient media, and to manage liquid and gaseous waste generated. The fluid controller may include hardware or software, including fluid control logic. An electronic controller may be included containing fluid flow electronic components to manage the application of liquids and gasses (e.g., CO₂, media, drug application, liquid and gaseous waste, etc.). The footprint of slide (e.g., the smart slide described herein) may be similar or identical to an SBS compliant, standard 6-well microtiter plate (each well capable of containing 5 mL of media volume).

The multi-well tissue culture plates described herein may be configured as micro-bioreactors. For example, each well may be referred to as a bioreactor (or a micro-bioreactor). In operation, the system may control one or more (e.g., 2, 3, 4, etc) sensors for detecting parameters of the wells/bioreactors and materials therein (including non-temperature parameters). For example, a first optical sensor may be used to detect pH, a second optical sensor may be used to detect pO₂ (e.g. dissolved O₂), a third sensor may be used to detect pCO₂, a fourth optical sensor may be used to detect pN₂, etc. Any or all of these sensors may be included or may be incorporated as part of each well. It may be particularly useful to operate each sensor independently. For example the well (or a region of a well) may be activated (e.g., by emitting light of a particular wavelength) to excites a first optical sensor (e.g., a pO₂ sensor). The response of the sensor (e.g., absorption and/or emission) is then detected by a detector, and the detected response from the sensor is analyzed. The response typically reflects the characteristic of the system or a parameter of the culture conditions within the well. A second round of activation/sensing/detection/analysis may then be performed on the same or a different sensor.

Product sensors (e.g., sensors for detecting a produced product) may also be included as part of the multi-well tissue plates, as described herein. A product sensor may be included as part of each well. For example, a product sensor may determine (e.g., by FRET, displacement of florescent binding, etc.) the binding of a product within the well. The product sensor may also include sensing logic to determine concentration based on the binding kinetics and/or florescence intensity.

Although it may be possible to activate, sense, detect and analyze multiple sensors at the same time, it may be desirable to activate, sense, detect and analyze each sensor individually. For example, individual sensing, detection and analysis may prevent cross-talk between sensors. Individual sensing also allows separation of testing and responding to individual parameters. This can be particularly useful when the sensor is used as part of a regulatory feedback loop, where the sensor can be specifically activated, detected and analyzed to control a certain specific feature (e.g., pH), without requiring detection or analysis of other features. Thus individual features (e.g., pH, dissolved gas, etc.) may be detected and/or regulated with different frequencies.

These and other embodiments, features and advantages will become more apparent to those skilled in the art when taken with reference to the following more detailed description of the invention in conjunction with the accompanying drawings that are first briefly described.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention.

FIG. 1 illustrates one variation of multi-well plate as described herein.

FIG. 2 illustrates a system including a multi-well plate as shown in FIG. 1.

FIG. 3 shows detail of one variation of a multi-well plate.

FIG. 4A shows another variation of a multi-well plate.

FIG. 4B shows a multi-well plate having stirring beads therein.

FIG. 4C shows variations of stirring beads, as described herein.

FIG. 5A shows a skin explant grown in an incubator (control), and FIG. 5B shows a similar skin explant grown in a well of a multi-well plate as described herein.

FIG. 6A shows an explant grown in an incubator (control), and FIG. 6B shows a similar explant grown in a well of a multi-well plate as described herein.

FIG. 7 illustrates one variation of the bottom of a multi-well plate, including a micro-heater.

FIGS. 8A and 8B show variations of the bottom of a multi-well plate.

FIGS. 9A and 9B show schematics of cell counters that may be used as described herein.

FIG. 9C shows different variations of light emitters that may be used as part of a sensor as described herein.

FIGS. 10A and 10B schematically illustrate a product sensor as described herein.

FIG. 10C shows an exemplary competition binding graph of fluorescently-labeled product.

FIGS. 10D and 10E show one variation of the product sensor described herein.

FIG. 11 illustrates one variation of a sensor array useful as part of a micro-bioreactor.

FIGS. 12A and 12B illustrate one variation of a product sensor as described herein.

FIG. 13A shows a side perspective view of one variation of a device as described herein.

FIG. 13B shows a cross-sectional view through one well of a device.

FIG. 14 shows an exemplary temperature profile across a glass plate that has been coated with ITO.

FIGS. 15A-C illustrate different variations of electrode arrangements for micro heaters as described herein.

FIG. 16A shows a cross-section though one variation of an inner shell of one variation of a multi plate device having six wells.

FIG. 16B shows a cross-section though one variation of a multi-well slide.

FIG. 17 shows one embodiment of a temperature sensor attached to a gimbaled mount, as described herein.

FIG. 18 shows an exploded view of a single multi-well slide, as described herein.

### DETAILED DESCRIPTION OF THE PREFERRED EMBODIMENTS

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. Hence, the invention is not limited to the preferred embodiments described exemplarily herein. Moreover, this description will clearly enable one skilled in the art to make and use the invention, and describes several embodiments, adaptations, variations, alternatives and uses of the invention, including what is presently believed by applicant to be the best mode of carrying out the invention.

As used herein, the terms “about” or “approximately” for any numerical values or ranges indicate a suitable dimensional tolerance that allows the part or collection of components to function for its intended purpose as described herein. Also, as used herein, the terms “patient”, “host” and “subject” refer to any human or animal subject and are not intended to limit the systems or methods to human use, although use of the subject invention in a human patient represents a preferred embodiment.

Described herein are multi-well plates including one or more on-board features for controlling or monitoring materials (e.g., media, tissue, etc.) in the wells of the multi-well plates, systems including multi-well plates, and methods of using these multi-well plates. Although many of the features are described and exemplified separately, it should be understood that these features may be included in any combination with other features, or other variations of the plates (including plates with only one well, or any reasonable number of wells).

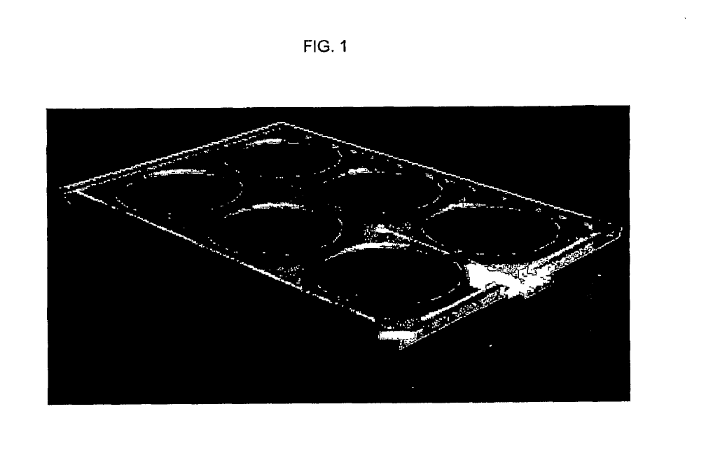

The smart well plates described herein may include any reasonable number of chambers or wells (e.g., 1, 2, 4, 6, 12, 18, 24, 48, 96, 192, 384, 768, 1536, 3072, etc.). FIG. 1 shows one variation of the smart multi-well plate described herein, having six independent chambers. The plate is compliant with typical (e.g., ‘standard’) sizes of cell culture plates. For example, the plate shown in FIG. 1 is SBS compliant (5.0 inches by 3.3 inches). However, any appropriate shape or size may be used. The wells may also be any appropriate shape and size. The six wells shown in FIG. 1 are 35 mm ID micro wells that are 19 mm deep. The sides of these wells are perpendicular to the bottom. The wells may also be any appropriate shape or size (e.g., volume). Typically, cells or tissue are cultured within the wells, thus the wells may be adapted so that the tissue or cells may attach or adhere in any appropriate surface. In some variations, the wells may be adapted to receive an insert containing the cells or tissue.

The multi-well plates (e.g., “microplates”) described herein may be used in biological and pharmacological research, and may be configured so that the dimensions (or other appropriate specifications) conform to industry standards. For example, the Society for Biomolecular Screening (SBS), began developing standards for mechanical uniformity of micro plates in 1995. In some variations, the devices described herein conform to the SBC standard footprint. For example, the plates described herein may be within the maximum dimension of the height requirements for micro plates established by SBS standards. In some variations, the plates may be handled by automatic handling.

Wells may also be treated or coated with any appropriate agent. For example, coatings to enhance cell adhesion or cell culture may be used (e.g., poly-L lysine, etc.), Or coatings to inhibit cell growth in some regions (e.g., the walls, around port openings, etc.) may also be used.

In FIG. 1, the bottom of the wells is made of a clear glass material, allowing the well to be visualized from beneath. In general, the smart well plates described herein may be made of any material or combination of materials. However, as described more fully below, it may be beneficial to have at least a portion of the well (and/or lid) comprise a transparent material that permits light of any desired wavelength (e.g., U.V., visible, etc.) to pass, permitting visualization and/or treatment (e.g., by exposure to light, such as laser light, or other radiation) of cells growing within the wells. Thus, at least a portion of the smart wells may comprise glass, ceramic, or polymer. For example, the wells may be at least partially made of a polymeric material such as PET (Polyethylene Terephthalate). Any appropriate material may be used. Different regions of the multi-well slides may comprise different materials, having different properties. For example, in FIG. 1, the sides of the wells are made of PET, while the bottom is glass. Furthermore the bottom has been coated with a material to permit thermal control of the wells, as described below in the section titled “Temperature regulation”.

The multi-well plates may also have a desired thickness in different regions. For example, the bottom of the multi-well plate (shown as glass in FIG. 1) may be thin enough to permit imaging through it using an inverted microscope (e.g., approximately 0.5 mm thick clear glass).

The multi-well plate may also include a removable lid. The lid may cover each well either loosely (as in standard multi-well plates) or it may seal over some or all of the individual chambers. For example, the lid may include one or more gaskets (e.g., o-rings) for sealing around the lip of the wells, preventing any uncontrolled exchange of material between them, and also helping to maintain the microenvironment formed within each well. The lid may also comprise one or more materials, and may permit light or other modes of sensing to pass through all or a region of the lid. For example, the lid may include a glass region, or other transparent and/or translucent region. Non-removable lids may also be included as part of the multi-well plate. As mentioned above, the lid may seal against the wells. The lid may be a single (unitary) lid covering all of the wells, or it may be divided into separate lids (e.g., separable operable lids covering one or more individual wells). The lid may secure onto the wells. For example, the lid may snap and/or lock onto the well(s).

The lid may also be heated, as described below in the Temperature regulation region. Heating the lid may prevent condensation, and may be used to regulate the temperature of a well in the plate (e.g., instead of or in addition to heating the bottom and/or sides of the plate or individual wells). In general the lid may include any of the features described herein as part of the multi-well slide (e.g., ports into/out of the wells, sensors, temperature controlling elements, data and/or power inputs/outputs, magnetic stirrers, etc.). In some variations, the lid maybe reused with different lower portions of the multi-well slide. For example, the lower portion may be disposable while the lid is reusable, and may be sterilizable. For example, the lid may be configured to allow sterilization (e.g., by heat, alcohol, radiation, etc.). This may save cost, as sensors, magnets, heating elements, etc. may be placed on the lid instead of the lower region of the multi-well slide. Accept where the context makes it clear otherwise, any of the features, uses or properties herein attributed to the “plates” or “multi-well plates” may be incorporated into the lid. For example, the ports, magnets (stirrer) or the like may be present on the lid and/or the lower region of the plates.

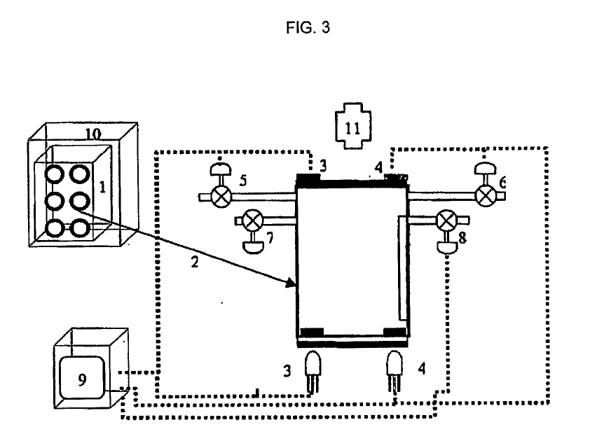

In FIG. 1, the multi-well plate includes multiple ports into and/or out of each well. In particular, FIG. 1 shows four ports accessing each of the six wells. However, any appropriate number of ports may be used (e.g., 1, 2, 3, 4, 5, 6, etc.), although typically at least two ports are included. Each port may include a dedicated function. For example, a port may be used for adding or removing material (e.g., media, gas, salts, etc) to and/or from the well. A port may also be used to extract samples from the well. FIG. 3, described further below, shows examples of four different ports of a multi-well plate including a feeder port 7, a sample port 8, a NaOH μ-pump port 5, and an O₂ port 6.

The ports shown in FIG. 1 are located on the side of the plate. One of the four ports for each well is spaced differently from the others, providing a reference so that a user can readily distinguish which port each one is. Similarly, ports may be different sizes or shapes, and may include quick-connect or quick-disconnect features. Ports may also include one or more valves controlling the opening/closing of the port. Valves may be controlled manually (e.g., by turning, pushing, etc.), or automatically (e.g., solenoid valves). Valves may also include emergency release mechanisms (e.g., overpressure release valves). The size of the different ports may be determined based on the purpose of the valve (e.g., for use with liquids, gasses, etc), the presence or absence of a filter as part of the port, and the desired range of flow rates through the port.

For example, a port may be provided for applying media (e.g., culture media) to an individual well of the plate. The outlet for the port within the well may be located in any appropriate position, including the upper portion of the wall of the well (so as not to disturb cultures growing at or near the bottom of the well), or near the bottom of the well (for more rapid exchange of culture seen by the cells or tissue in the well). In some variations, the wells may include ports connecting adjacent wells to each other, allowing passage of material between individual wells of the plate.

Flow of fluids (liquids, gases) into or out of the wells through the ports can be controlled (instead or in addition to the valves) by one or more pumps, or by gravity feed. Any appropriate pump may be used (e.g., peristaltic pumps, etc.).

One or more filters may be included with the port to filter material entering or exiting the wells through the ports. For example, a filter may prevent contamination by preventing cells (e.g., bacteria, etc.) from pasting through the port into or out of the wells. Filters may also prevent particulate matter from entering the port or the well. For example, in variations including the magnetic stirrers (described further below), a filter may prevent magnetic stirring particles from leaving the wells of the plate. Any appropriate filter or type of filter may be used, including fibrous filters, porous membrane filters, capillary filters, fabric filters, etc. Selective filters (e.g., size selective filters and active selective filters such as electrostatic filters) may also be used.

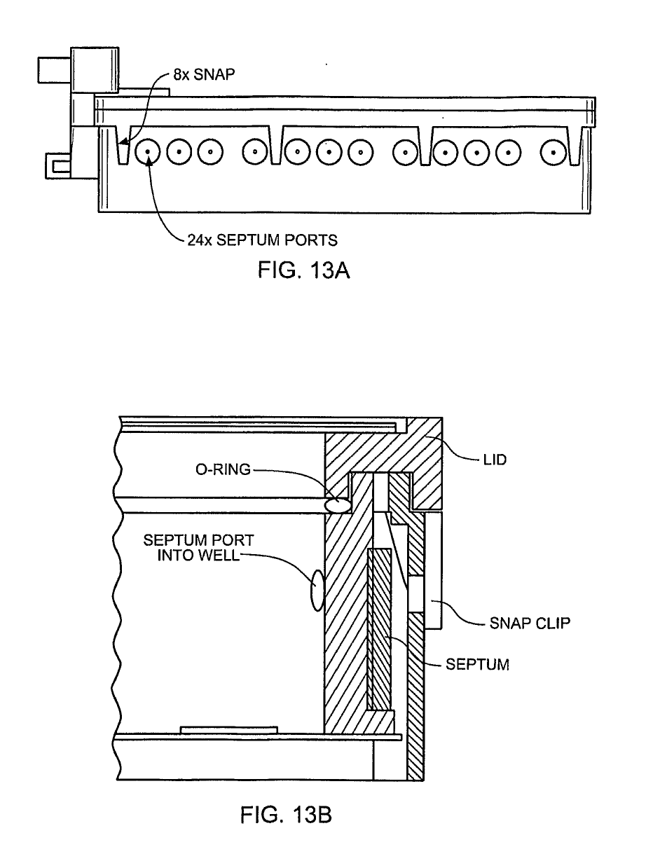

As described above, each well may have one or more sample port, which may be used to remove material from within the well. A sample port may be configured so that material from within a well may be removed without breaking the biological isolation (e.g. seal) on the well. For example, a sample port may be configured as a septum through which a sampling device (e.g., a needle) may be inserted to remove material. In some variations the septum comprises a material, such as an elastomeric material, that seals around the sampling device inserted through it, and closes back up after removal of the sampling device. For example, the septum may comprise an elastomeric plug (e.g., a plug made from rubber, silicone, etc.). FIG. 13A illustrates a side perspective view of one embodiment of such a device. In this example, multiple septum ports are aligned along the outer wall (near the upper edge) of the multi-well plate. Each septum provides access to a single well. FIG. 13B shows a cross section illustrating additional features that contribute towards the biological isolation of each well of a multi-well plate as described. For example, in FIG. 13B, the septum port is shown passing through the upper region of the wall of a well. The septum port on the inner side of the wall is contiguous with the septum on outer wall, providing access through the septum material into the well. In this example the septum port in the well is approximately 17 mm above the bottom of the well, however it should be understood that the septum port may open into the well at any appropriate height or position. For example, it may be beneficial to access the well from the bottom or middle region.

FIG. 13B also illustrates in cross-section a multi-well plate in which a lid has been sealingly engaged with the well. The lid is shown as closing over the well and pressing against a seal (shown here as an O-ring) around the inner lip of the lid-engaging region of the well. Furthermore, the lid shown in FIG. 13B also includes snap clips that extend from the lid to engage snaps (receptacles) on the body of the multi-well device. In some variations the snap clip extends from the body and engages receptacles on the lid. In general, the snap clip engages the lid so that it may be sealed against the well, isolating it from the external environment, and from adjacent wells.

The plates may also be connected to one or more power sources. In FIG. 1, the plate includes an onboard power connector providing power to the heating portion, any valves, any controlling logic (e.g., hardware) on the plate. In some variations, power may be supplied by a power regulator or power controller. In some variations, the power may be supplied by an onboard power supply, such as a battery. The plate may include power conditioning circuitry to regulate power to the various components, including the heater. Separate power supplies may be provided to different components of the plate (e.g., heater, sensors, valves, etc.).

As described further below, any appropriate sensor may be incorporated into the plates. Sensors may be positioned either within one or more of the wells, outside of the well, or both inside and outside of a well (for example, embedded within the body of the plate). Optical sensors (e.g., for detecting temperature, pH, dissolve gasses such as O₂, CO₂, etc.) are particularly useful since at least a portion of the plate may be made transparent to the sensor, so that it may detect changes within the environment of the wells without disturbing the well chamber. Any appropriate type of sensor may be used.

### Temperature Regulation

Any appropriate heating or cooling components may be used. For example, the temperature of one or more wells of a plate may be heated by a heat-generating device such as a micro-heater comprising a combination of electrically conductive coating applied to a region (or regions) and one or more electrodes arranged to contact the electrically conductive coating. When electrical energy is applied across the electrically conductive coating, resistive heating warms the region of the plate covered by the electrically conductive coating The arrangement of the electrically conductive coating and electrodes may be configured to allow accurate and/or uniform resistive heating of the wells of a plate. For example, each well of the plate may be controlled to within ±0.1° C. over the range of 30° C. to 60° C. An example of micro-heating similar to the micro-heater described herein for use with the multi-well plates may be found in WO 2005/118773 (titled “Apparatus and Methods for Multiplex Analyses”) which is herein incorporated by reference in its entirety.

Micro-heaters are typically made of coatings of materials having high thermal conductivity and chemical stability. Such materials include, but are not limited to metals such as chromium, platinum and gold, and semi-conductors such as ceramics, silicon, and geranium. A material that is particularly suited to form the heating coating is indium tin oxide (ITO). ITO is a transparent ceramic material with a very high electrical conductivity. ITO can be layered (e.g., by sputtering, thermal evaporation, etc.) onto the substrate of the plate. The coating may be placed in electrical contact by an electrode (or multiple electrodes) to form the heating element. When current is passed through the coating, the coating heats up. The heating element is typically connected via electrode contacts to electrical leads which connect to a power source that provides voltages agrees the element and effects subsequent heating. The heating element may also be coupled to a temperature sensor to control the temperature and hence the thermal profile of the plate. Furthermore, the electrode contacts may be configured to provide uniform and controllable heating across the plate. The electrical contacts may therefore be distributed so that the current density across the ITO coating is uniform, or is distributed to prevent heating some regions more than others. For example, in some variations, the electrode contacts are distributed in a tapered fashion across the base of the plate after it has been coated with ITO. Examples of the tapered electrodes which may be used to warm the plate include electrodes that are wedge-shaped, curved, and vary in thickness or angle.

Other conductive (heating) coatings that are substantially transparent include any transparent conductive oxide such as ITO, FTO (fluorine-doped tin oxide), ATO (antimony tin oxide), etc. Any appropriate method may be used to apply the conductive coating, including sputtering, printing, painting, dipping, or the like. For example, ATO may be applied using a film patterning technique, such as film patterning using and inkjet to form the heater/electrode arrangement. Thus, ATO may be “printed” instead of sputtered by using ATO particles in a liquid medium (e.g., a suspension). The coating (e.g., ATO) can be annealed at low temperature in a vacuum after printing. ATO may be particularly useful, since is typically over 95% transparent (compared to ITO's 85% transparency in the visible region). The electrical connection (e.g., electrode) may also be formed by printing, sputtering (e.g., masking), etc.

Transparent conductive coatings may allow the well to be visualized or imaged through the heating element. Thus, any relatively transparent conductive films may be used, including, e.g., conductive cadmium oxide (CdO) films.

In some variations, the micro-heater includes a protective layer of material to protect the conductive coating. A protective layer may be placed directly against the resistive coating. Thus, the protective layer may comprise a clear (light-transmitting) layer. In some variations, the protective layer is a protective coating. For example, a coating of SiO may be used to protect a conductive coating (such as ITO). As mentioned above, coatings (including the conductive coating and a protective coating or coatings) may be applied in any appropriate manner, including dipping, painting, sputtering, spraying, evaporating, etc. In some variations, the protective layer may also be a thermally (and/or electrically) insulative layer.

Any appropriate region of the plate may include a conductive coating (e.g., the bottom, sides, lid, etc.). Coatings are preferably done external to the inner surface of the well (which will not contact the tissue or cells within the well). Coatings may be external, or they maybe located (e.g., sandwiched) between other regions of the plate. For example, a conductive coating may be covered with an insulating and/or protective coating as just described. For example, thermally and/or electrically insulating materials are particularly useful for protecting the micro-heating region.

As mentioned previously, one or more thermal sensors may be included to help regulate the temperature of the plate when using the micro-heating method described. For example, a thermal sensor may be connected to a controller either on the plate (e.g., integrated into the plate) or off of the plate (e.g., connected to a controller) to provide feedback to regulate the plate temperature. The temperature sensor (or sensors) may be located in any appropriate location of the plate, and therefore may help regulate the temperature at any appropriate region of the plate (e.g., the wells). For example, in some variations, the temperature sensor is located adjacent to a wall of one or more wells in the plate. This may allow the temperature to be regulated based on the temperature of the media or material within the well. In some variations, the temperature sensor(s) is preferentially located near the electrically conductive coating.

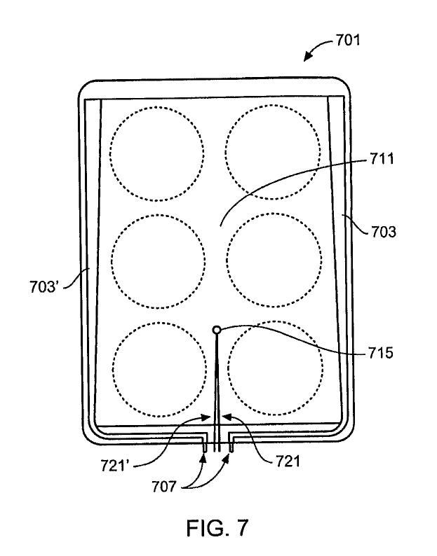

FIG. 7 illustrates one example of a multi-well plate (shown as a 6-well plate) that is temperature controlled. In FIG. 7, a region of the bottom of the plate 701 is shown coated with a transparent coating of ITO (electrically conductive coating) 711. The ITO coating 711 is also connected to two tapered electrodes 703, 703′ on both sides of the plate. The narrower end of the tapered electrodes 703, 703′ are connected to a voltage/current source (not shown) through connectors 707. The connectors 707 (or a single connector connecting both electrodes), are shown projecting from one side of the plate. Although a pair of connectors corresponding to each tapered electrode is shown, a single connector may be used.

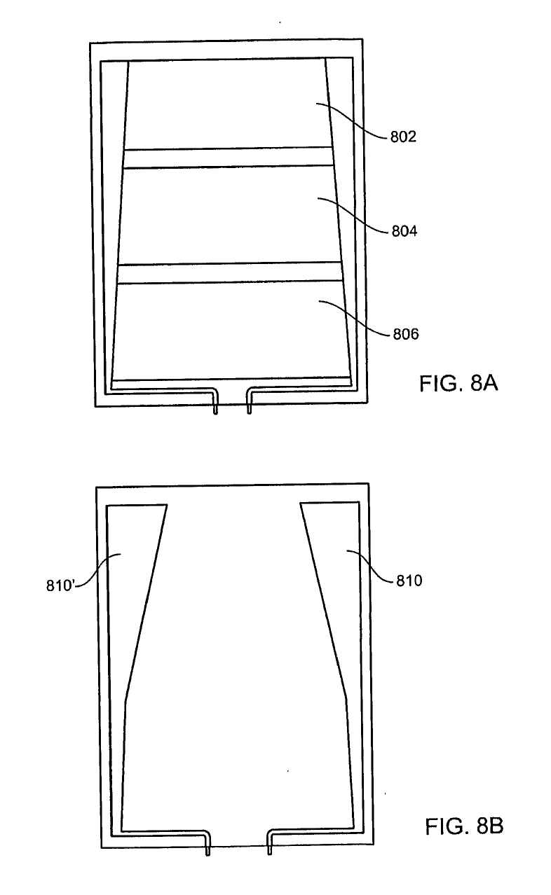

As described above, any appropriate electrode may be used to contact the electrically conductive coating. For example, the electrodes 703, 703′ shown in FIG. 7 are metal electrodes that are applied to the bottom of the plate in a tapered pattern that is triangular, with the wider region of the electrode furthest from the connector contact. Metal electrodes such as these may be applied by printing, painting, adhesive, etc. Thus, since current passes from the electrode from the direction of the connector contact, the current density may decrease. The tapered shape of the electrode allows the current heating the ITO coating to more controllably heat the plate. Any appropriate ‘taper’ may be used, including non-uniform tapers 810, 810′, as shown in FIG. 8B. The taper shown in FIG. 7 and FIG. 8A changes from a narrow diameter (approximately 2 mm near the connector) to a wider diameter (approximately 4 mm furthest from the connector) at a constant (straight-line) rate. In some variations, the electrode is tapered by changing the depth (e.g., thickness) rather than (or in addition to) the width, as shown in the figures. The geometry chosen (e.g., the shape of the tapered electrode) may determine (in part) how the temperature is distributed across the plate. For example, the taper may be chosen so that the temperature between different wells that are all connected to the same electrodes is approximately equivalent (e.g., within ±1° C., ±0.5° C., ±0.1° C., etc.). In some variations, the temperature may be distributed non-uniformly between different wells. For example, the temperature may be slightly cooler in the wells closer to the connector (e.g., 36.5° C.) compared to the end of the plate further from the connector (e.g., 37° C.).

The electrically conductive coating 711 shown in FIG. 7 is uniformly spread across the entire bottom surface of the plate. Thus, a single coating region contacts both electrodes. In some variations, different regions of the plate may correspond to different electrically conductive coatings, as shown by FIG. 8A. In FIG. 8A, three separate electrically conductive coating regions are shown 802, 804, 806. These regions are each electrically connected to the electrodes printed on the bottom of the plate. In some variations, each region is connected to an individual electrode (or set of electrodes). These regions may correspond to one or more wells. For example, each of the three regions shown in FIG. 8A corresponds to the base of two wells (not shown).

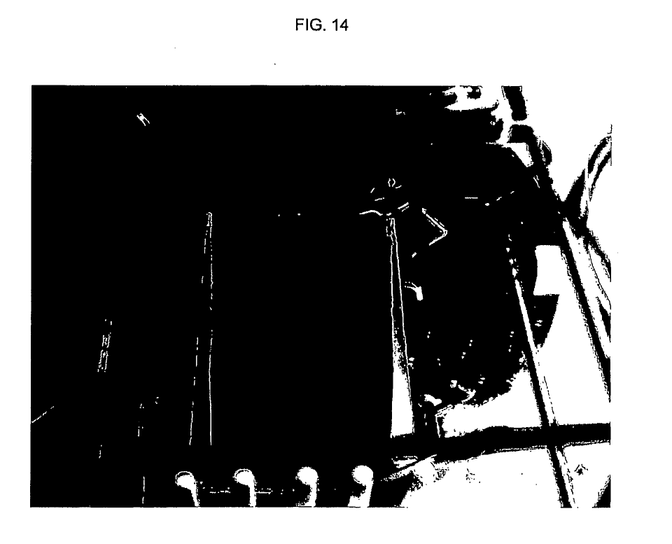

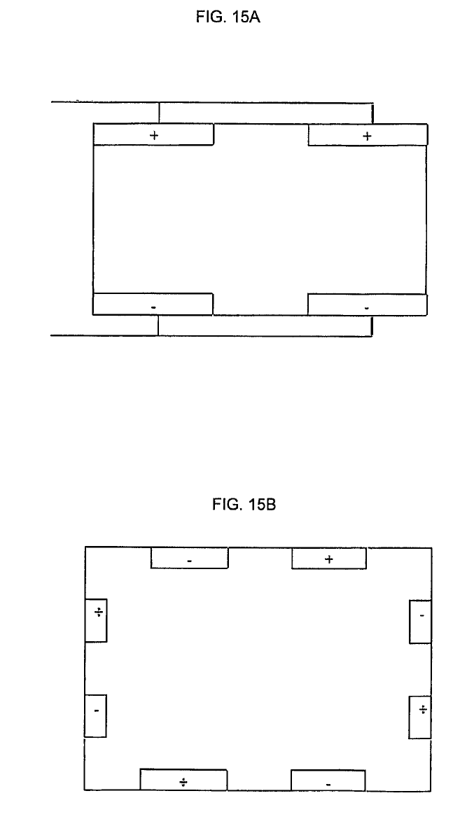

In general, uniform temperature control both within a well, and across different wells of a plate, is desirable, and may be accomplished using appropriate electrode and coating regimes, as described herein. For example, FIG. 14 illustrates the temperature profile across a glass plate that has been coated with ITO. The ITO is in electrical contact with two tapered electrodes (similar to those shown in FIGS. 7 and 8). The ITO-coated glass may serve as (or be thermally connected to) the base of the plate or a top for the plate. In this example, the glass plate is a large thermal mass having a large surface area for heat radiation. The outer corners tend to cool, while the inner center region becomes warmer than the outer edges, resulting in a temperature gradient. When this system is used as a micro-heater for the base of the plate, more extreme non-uniform heating may affect the temperature within different wells. In FIG. 14, a thermal plastic sheet is used to demonstrate regions of different temperature in one variation of a heating system. Although these differences may be slight, and confined to the periphery of the plate, it would be desirable to eliminate them.

In some embodiments, different arrangements of transparent electrically conducive material may be used to generate heat across the plate. For example, FIG. 8A (described above) shows one variation of a heating plate having different heating zones traversing the longest dimensions of the plate. In one variation of this design, a series resistor may be connected between the center zone and the source of electrical energy.

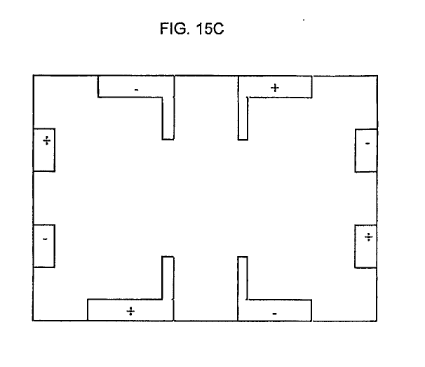

In other variations, the shape and positions of the electrodes may be configured to produce a more uniform distribution of temperature across the plate. For example, in FIG. 15A, a micro heater comprises four electrodes (two on either long axis of the ITO coating. The “gap” in the electrode coverage along the long axis of the device may cause a reduction in heat in the center of the plate. FIG. 15B illustrates another variation of the device, in which the periphery of the ITO coating includes electrodes of alternating polarity. This arrangement may allow for increased heating at the edges and corners, so that heat is not exclusively concentrated at the center region. FIG. 15C shows a design in which the electrodes extend from the periphery of the ITO coating towards the center of the device (resulting in the “L”-shaped electrodes shown). The polarity of these electrodes alternates. In this variation, the electrodes can still be kept clear of a base region of a particular well, allowing visualization through the base of the well. In some variations, the electrodes may be at least partially visible through the well of the plate. As mentioned previously, the electrodes may comprise any appropriate material. In some variations, the electrodes comprise flex circuit connectors. For example, flex circuit connectors may be made from polyamide with two or more layers of copper.

Of course, a combination of the different region so transparent electrically conductive coatings and electrode contact may be used, as described briefly above.

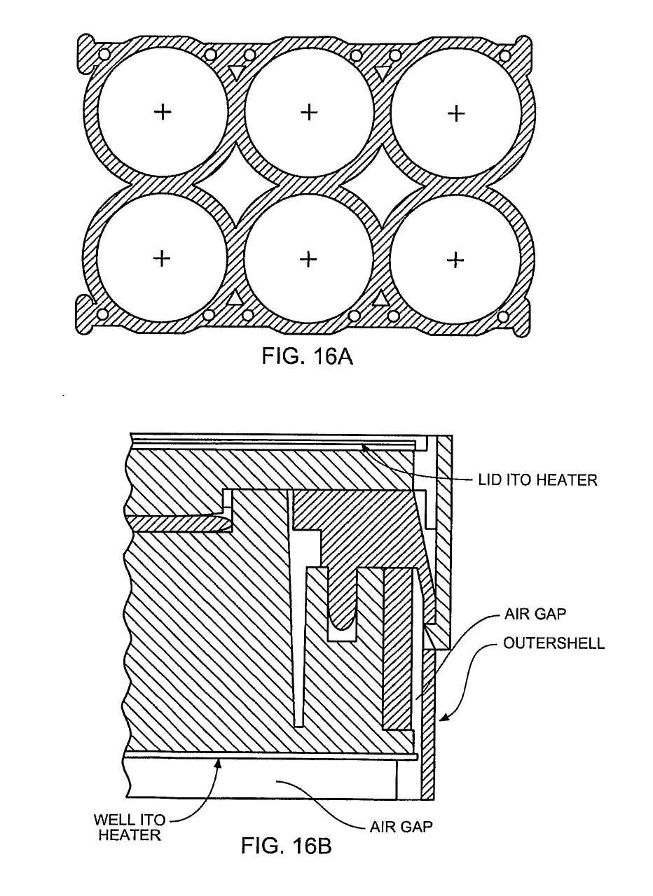

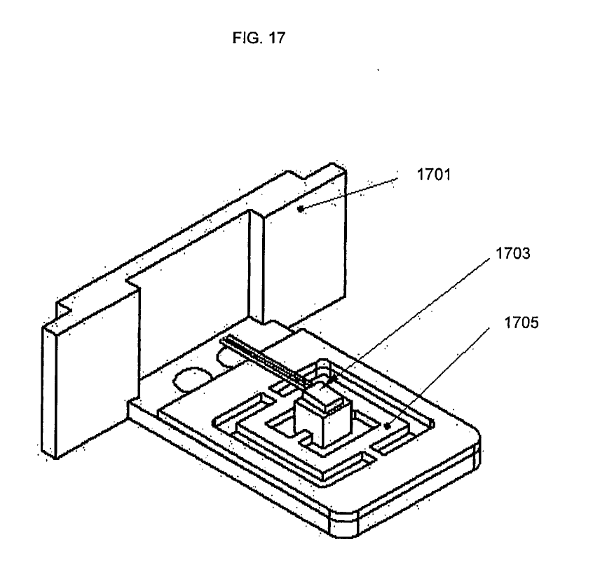

In some variations, the plate (e.g., individual wells of the plate, or portions of the wells) are thermally isolated by the inclusion of an air gap surrounding the plate or each well. An air gap helps to thermally isolate the wells from the surroundings. For example, the base of the plate may rest on a metal surface, or a surface that would otherwise sink heat from the plate (by thermal conduction). Thus, the base of the plate may be isolated from this kind of thermal conduction by including an air gap between the base (or sides) of the plate and the surface upon which the plate rests. In some variations, the plate comprises an inner shell that may be surrounded by an outer shell. The inner shell may form one or more of the walls of the wells. FIG. 16A shows a cross section of one variation of an inner shell having six wells. A base plate (configured as a heater, by coating with thermally-conductive material connected to electrodes, as described above) is connected to this inner shell, and the inner shell is surrounded by an outer shell. In some variations, the outer shell and the inner shell meet at discrete points, minimizing the thermal contact between them. In some variations, these contact points are insulated by a thermal insulator.

FIG. 16B shows a cross-section though one variation of a multi-well slide, showing the inner shell connected to the outer shell. Air gaps are visible between the inner shell and the outer shell. In FIG. 16B, a lid (also having a heater) is also sealed over the plate. Other variations having an inner and outer shell are shown in more detail below, in the section on Making or Manufacturing Multi-Well Plates.

As described above, at least one temperature sensor 715 may be in contact with a portion of the plate. In FIG. 7, the temperature sensor is a Resistance Temperature Detector (RTO) that contacts the ITO coating near the connectors. Two electrical leads 721, 721′ contact the RTO and attach to another connector 725, so that the temperature may be determined and may feed back into the regulation of the plate temperature, as described above. More than one temperature sensor may be used, and the temperature sensors may be used in any appropriate location.

In some variations, a temperature sensor is connected to the outside of a well at or near the heater substrate (micro heater) bottom of the well. A temperature sensor may be in thermal communication with a portion of one or more wells of the plate by either directly contacting a wall of the well (e.g., the outside of a glass plate forming the bottom of the well), or by contacting the well through an intermediary. For example, the temperature sensor may communicate with the heater substrate of the well through a thermally conductive grease or oil. By using the oil or grease as an interface, the temperature sensor does not need to directly (physically) contact the glass substrate, preventing scraping or other types of mechanical damage to the plate.

Mechanical damage to the outer surface of the well (or to the temperature sensor) may also be prevented by mounting the temperature sensor to a gimbaled mount. In general, a gimbaled mount has one or more rotation degrees of freedom, adjusting the position of the temperature sensor until it is in contact with an outer portion of the well, or a glass region forming the bottom or top of a well. The gimbaled mount may flex or rotate to allow the temperature sensor to rest against the glass without damaging it. FIG. 17 shows one embodiment of a temperature sensor attached to a gimbaled mount having two degrees of removable freedom. This gimbaled mount may be configured as a removable mount. In FIG. 17, the gimbaled mount includes a bracket 1701 to which the base 1705 is connected at a 90° angle. The bracket is configured to mate with a region of the plate (e.g., a region of the outer shell) and allow the temperature sensor 1703 (Resistance Temperature Detector or RTD) to contact the glass substrate coated with thermally conductive material that forms the base of the plate. The gimbaled mount may be made (at least partly) of an elastomeric material or a material that is relatively flexible. In some variations the gimbaled mount comprises an injection molded polymeric material. In the variation shown in FIG. 17, the mount comprises cut away sections that allow the mount to move (e.g., bend or rotate) readily. The concentric support rings are each connected at two points, allowing movement at the unconnected regions.

As mentioned above, temperature sensors as described herein may be removable from the plate, and may be used with other plates. Thus, even if the micro plates described herein are disposable, some components of the plates (e.g., the temperature sensor) may be reusable. For example, the gimbaled mount described above may be a removable gimbaled mount. The gimbaled mount may make it easier to disengage and re-use a temperature sensor on other plates (or other portions of a plate) without damaging the plate, the mount, or the temperature sensor.

The lid (or top) of the plate may also include a similar temperature control (e.g., electrically conductive coating and electrode, as well as one or more temperature sensors). The connectors described for the temperature sensor and the electrodes may all connect to a controller, as described in more detail below, for controlling the temperature of the plate (e.g., the wells and/or the lid or top of the plate).

In addition to temperature sensors that may be detect and help control the temperature of the plate, other sensors may be used as well.

### Sensors

Any appropriate sensor may be used to help monitor and control the environment within one or more of the wells of the plate. In general, the sensors may include non-invasive sensors (e.g., optically-based sensors, or sensors that detect properties from outside of the wells of the plate), and contact-type sensors, that may be located within the well, and may contact the cells and/or culture media within the wells. In some variations, sensors located within the wells may be “read” (e.g., sensed) by one or more transducers located outside of the wells, preventing contamination. Thus, the integrity of the well may be maintained when reading or using the various sensors. Sensors may particularly include sensors for detecting or sensing non-temperature parameters (as described in more detail below). It should be clear that non-temperature parameters include parameters that may be affected or modified by temperature (and thus may reflect temperature or have temperature information extractable therefrom).

Examples of typical sensors (including non-temperature sensors) may include pH sensors (e.g., off-the-shelf pH sensor such as phOptica, distributed by WPI and manufactured by PreSens Precision Sensing GmbH, and sensors from Fluorometrix™ may be used), dissolved O₂ sensors (e.g., OXYMINI/OXYMICRO sensors distributed by WPI and manufactured by PreSens Precision Sensing GmbH, or Fluorometrix™ sensors), optical sensors (e.g., optical imaging microscopes, cameras, etc.), cell counters (e.g., commercially available cell counting system such as Applitek™'s cell counting system). U.S. Pat. No. 6,673,532 and U.S. Pat. No. 6,602,716 (incorporated by reference in their entirety herein) describe additional examples of sensors and systems including sensors; this description is not limited to any particular type of sensor.

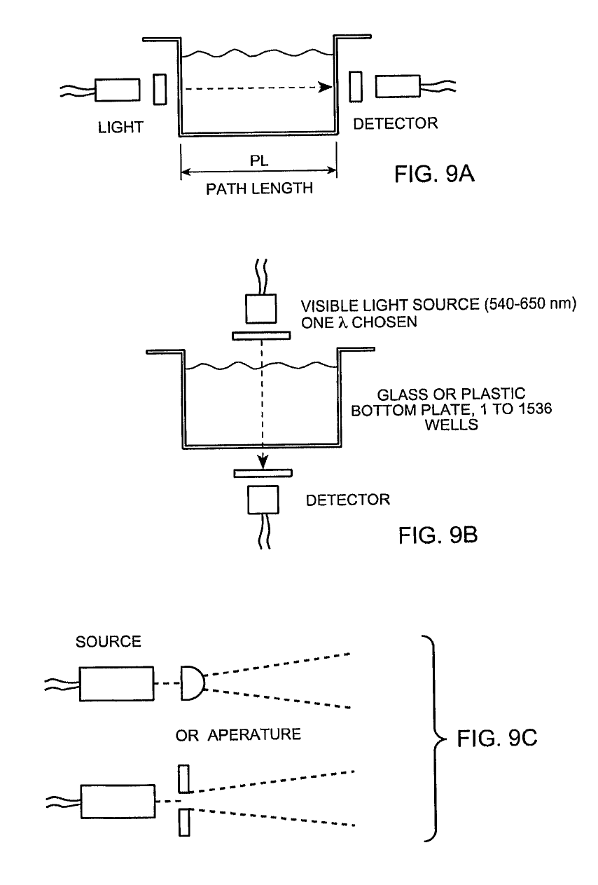

FIGS. 9A and 9B show different variations of a cell counter that may be used as one of the sensors described herein. For example, FIG. 9A shows a schematic of a side-on optical cell counter that may be used with the plates described herein. One (or all) of the wells may pass light (e.g., at a selected wavelength, λ). Thus, the well may include windows (to allow light to pass through it), or it may be at least partly transparent, at least to the wavelength(s) used to count cells. In operation, the cell counter may pass light from a light source, through the solution including the cells, where it is detected by a detector. The light may pass through a known path length (PL). The concentration of cells in the well may be derived from the relationship:

$$\frac{( F )}{Ab} = B * C . \qquad [ 1 ]$$

where (F) is a factor for the cell type to be determined (that may be determined experimentally (for example, by constructing a calibration curve based on counting with hematocytometer), Ab is the absorbance (e.g., at a particular wavelength, λ), B is the path length (PL) and C is the concentration of cells. Based on this relationship, it is apparent that the sensitivity of the cell counter doubles with a doubling of path length. For example, if we assume that the factor F is 0.001 for a cell type, and if there are 1000 cells/ml, then for a path length of 1.0 cm, the absorbance is 1.0; when the path length is 2.0 cm, the absorbance is 2.0.

Any appropriate light source may be used as part of the cell counter. For example, the light source may be a lamp, a laser, a diode, etc. The light source may be incorporated as part of the plate. The detector may also be incorporated as part of the plate. In some variations, both the light source and the detector are incorporated as part of the plate. The light source may include a lens to spread or focus the light and thereby increase the region illuminated; a lens may also be used on the detector. In some variations, an aperture may be used as part of the light source or the detector to limit the region of the light detected or emitted. FIG. 9C shows two different arrangements of emitters and detectors that may be used as part o the cell counter.

A cell counter with turbidity (light scattering) may also be used as part of the cell counter. This method may be particularly appropriate when measuring cells in suspension. Measuring light scattering to determine cell concentration may be derived because absorbance of some wavelength(s) of light may be proportional to the concentration (e.g., number) of cells in a well. For example, absorbance (Ab) may be expressed as:

Ab=−LOG(Transmittnce) [2]

where the incident light/transmitted light is related by the equation:

$$Ab = - LOG ( \frac{{I}_{o}}{I} ) \qquad [ 3 ]$$

A reasonable wavelength of light that can be used is between about 540 and 650 nm. As the density of cells increases, the absorbance will increase. FIG. 9B shows another arrangement of a cell counter in which the detector is located below the well of a plate, and the light source is located above, so that light passes through the well from the top to the bottom. As a selected wavelength of light passes through the liquid in the well, light is absorbed depending on the concentration of cells. The detector typically measures absorbance at the particular wavelength (e.g., 540 nm). The beam size may vary to match (or fit with) the size of the wells in which the cells are being measured. Further, the high of the liquid may also be chosen to optimize (or improve) the path length (since the absorbance may improve as path length increases).

Different variations of the cell counters or sensors are also possible. For example, the cell counter may be part of the plate (as described above), or the plate may be adapted for use with a cell counter or reader. For example, the plates may include a window of transparency through which the cell counter may read cell concentration. As shown in FIGS. 9A and 9B, the cell counter may operate in any appropriate direction, including through the well from the top to the bottom (or vice versa) or through the sides of the well.

Product Sensors

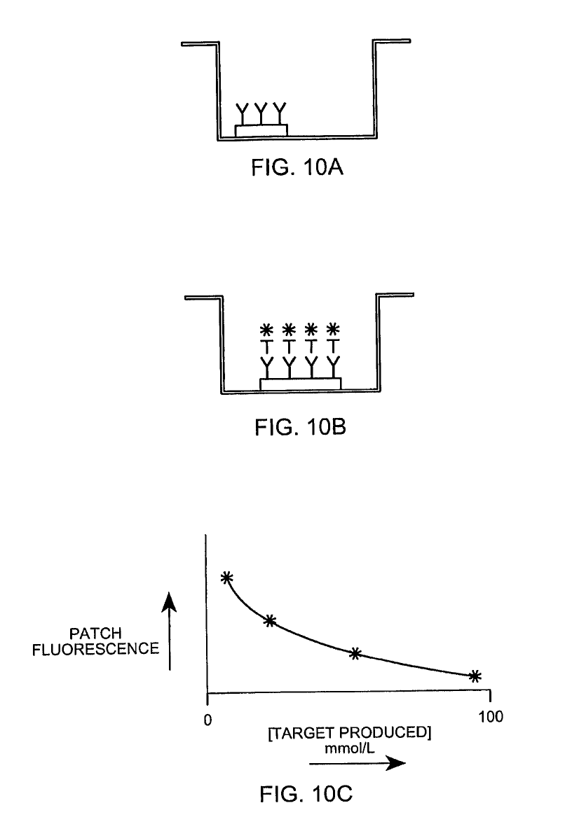

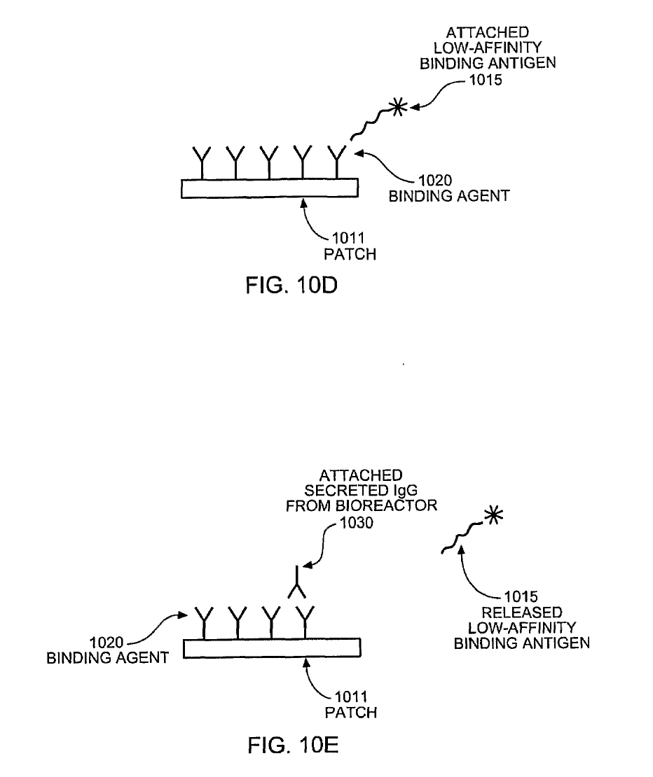

Sensors may also be used to determine the concentration of material released from cultured cells or tissue (e.g., by secretion, lysis, secretion, etc.). For example, a sensor may determine the concentration of proteins, hormones (e.g., steroids), nucleotides (e.g., DNA, RNA, etc.), carbohydrates, lipids, etc. Sensors may be electrical (e.g., electrochemical), optical, or chemical. For example, a sensor may be enzymatic, and include an enzyme (including a localized or tethered enzyme). In one variation the sensor determines concentration by an optical immunoassay. FIG. 10 illustrates one variation of an optical immunoassay that may be used as part of a sensor with the plates described herein.

A patch immunoassay may be used to detect (in real time) the presence or concentration of a product, for example, when the plate described herein is operating as a mini-bioreactor. Product (e.g., proteins, steroids, etc.) may be excreted or released from the cells, or the product may be expressed on the outer surface of the cells (e.g., the cell membrane). A region (e.g., a patch, spot, ring, etc.) within the well may be configured as a sensor to detect the product by coating an antibody (e.g., a monoclonal antibody) directed against the target. A sensing agent (e.g., an antibody) may be bound to a material (e.g., nylon, glass, etc.) within the well, such as the bottom of the well, or a region of an insert within the well. The antibody may be coated or bound by any appropriate method (as well known in the art of immunoassays), including binding to a secondary antibody (which may provide some amplification of the signal).

FIG. 10A shows a binding agent configured as an antibody that is bound to a spot within the bottom of the well. For example, this antibody may be a monoclonal antibody directed against the product (or a region of the product). The antibody is attached to the inside of the glass or plastic wall of the bioreactor well (e.g., multi-well culture plate). Standard coating techniques may be used, including sucrose preservation. After the antibodies have been attached to the spot so that the binding domains (arms) may still bind to target (e.g., product), purified displaceable target molecules which include an indicator such as a fluorescent molecule (fluorophore) are bound to the antibody, as shown in FIG. 10B. Any appropriate indicator may be used. In particular, fluorescent molecules may be used, particularly fluorescent molecules that do not readily “bleach” (e.g., lose their fluorescence). The displaceable target molecules may be bound by the antibody with the same affinity as the product (target) molecule, or the antibody may have lower binding affinity for the displaceable target molecule. This provides “loaded” product-sensors that may be incorporated into the well for optically detecting the presence of product. These loaded detectors may be dried and reconstituted within the well before use. In some variations, the detectors may be equilibrated.

In operation, the sensors detect the presence of the target product based on the binding kinetics of the target product to the antibody. As target product is produced by the cells within the well, the target product displaces the fluorescently labeled displaceable targets that were pre-loaded onto the sensor. Thus, the fluorescence within the well can change depending on the concentration of the product made by the cells. The presence of unlabeled target product in the bioreactor will displace the florescent target bound to the antibody, dispersing the florescent marker into the well (mass action). Displacement of the displaceable fluorescently labeled product is inversely proportional to binding of the unlabeled target product. Thus, the florescence of the spot or region will decrease as more unlabeled target binds to the antibody, in a concentration-dependent manner. FIG. 10C illustrates this relationship.

FIGS. 10D and 10E illustrate another variation of the sensor configured to detect product. In FIG. 10D, a sensor patch region 1011 is shown with an antibody attached. As described above, this patch immunoassay takes advantage of the binding kinetics of the product (or products) formed in the bioreactor, and is capable of real-time detection and monitoring. In some variations, the sensor may benefit from begin present within the bioreactor. For example, since the sensor takes advantage of the kinetics of binding of the product, the use of the sensor directly within the bioreactor allows many of the other properties of the bioreactor to enhance the sensor function. For example, the bioreactor environment may be temperature controlled, and the geometry (e.g., small volume) of the bioreactor, as well as the ability to provide mixing within the bioreactor further enhance the function of this sensor, and may also allow continuous monitoring.

In FIG. 10D the sensor (patch immunoassay) comprises a binding agent that is immobilized (e.g., fixed) to the bottom of the patch. The binding agent 1020 is at least partly free to bind a target. In the example shown in FIGS. 10D and 10E, a reporter molecule (1015) is a florescent marker that is attached to a low-affinity binding molecule. The marked low-affinity binding molecules are pre-loaded onto the binding agent/sensor. As described above, when a newly secreted target molecule (e.g., an IgG molecule) formed in the bioreactor is secreted, it out-competes the low-affinity binding molecule, and binds to the binding agent 1020, releasing the reporter 1015 into the bioreactor, and altering the florescence signal from the sensor patch region, as well as the florescence in the bioreactor solution. This change in the florescence (including the distribution of florescence) is detectable, and can be correlated to the concentration of target, based upon this displacement.

Any appropriate binding agent 1020 may be used as part of a sensor. For example, the binding agent immobilized as part of a patch immunoassay may comprise an IgG species IgG antibody, such as an a-mouse or an a-human IgG antibody. In one variation, the binding agent is a Protein A staphylococcal or recombinant (e.g., an IgG specific binding agent). The binding agent may be a Protein G (e.g., E. coli or recombinant) Fc binding agent. In one variation, the binding agent comprises a Protein A/G prom bacilius or recombinant that binds all human IgG subclasses. Thus, the binding agent need not be an antibody directed against the specific target molecules produced in the bioreactor. As described, the binding agent may be broadly directed against any antibody, or subtype of antibody (e.g., human or mouse IgG).

This patch immunoassay sensor technique thus allows for continuous monitoring of product formation within a well of the bioreactor. Any appropriate detector may be used with the product sensor described. For example, a fluorescent emitter and detector (capable of detecting the fluorescent marker on the displaceable target molecules) may be used. In some variations, the product sensor immunoassay (e.g., the antibodies) is bound to a transparent substrate so that they may be imaged through the well. It should be clear, however, that the substrate does not need to be clear, and the antibody may also be bound to a non-transparent substrate.

As described above, an immunoassay (patch) product sensor may be used to detect hybridoma product expression, including real-time detection. A patch immunoassay may be configured to monitor many different hybridoma products, including (but not limited to) proteins such as antibodies, insulin, cytokines, etc.

A patch sensor may include any appropriate immunoassay. For example, the detection format may be based on a solid phase type assay, a liquid phase binding type assay, or a hybridoma product. For example, in a solid phase assay, a product-binding partner may be bound to a substrate (e.g., the “patch” at the base of a well). The binding partner may be specific (e.g., a product-specific antibody), or a general product binder. As described above, the amount of product may be determined by displacement of a labeled competitive binder. In one variation, a capture element product binding partner) is pre-loaded with a fluorescently labeled product-competitive binder. For example, the patch may include a non-florescent antibody bound to the patch or bound to a bead (including a florescent bead). This first-layer antibody may be an anti-idiotype antibody (to bind any produced antibody) or a product-specific antibody (e.g., an anti-insulin antibody). The first-layer antibody is pre-loaded with a labeled competitive binder that binds with the first layer antibody. Thus, if the first-layer binder is an anti-insulin antibody, the labeled competitive binder may be fluorescently labeled insulin. If the first-layer antibody is goat anti-mouse IgG antibody, then the labeled competitive binder may be fluorescently labeled IgG.

In this example, as product is formed in the bioreactor it will bind to the capture element on the solid phase and displace the florescent labeled product. The total florescence of the patch will therefore decrease as more of the product binds. In general, pure hybridoma product will displace the labeled competitor in a dose-responsive manner. The bound florescent label may be displaced because it has a lower binding affinity. For example, the labeled competitor may experience steric hindrance because of the marker, or it may have a lower binding affinity for the capture element (e.g., because of the increased size of the marker, or because it is a different molecular sequence).

A product sensor may also be based on detection of Florescence Energy Transfer (FRET) type assays. FRET immunoassays involve the transfer of energy from a donor molecule to an acceptor molecule. FRET typically detects when a molecule that is fluorescently labeled with a first fluorophore is brought into close proximity to a molecule that is fluorescently labeled with a second fluorophore. If the excitation profile for the first fluorophore overlaps with the emission profile of the second fluorophore, then when the two labeled molecules are in close proximity (e.g., typically within less than 0.01 μm), excitation of the second fluorophore will cause excitation of the first fluorophore. Likewise, if the excitation profile for the second fluorophore overlaps with the emission profile of the first fluorophore, then excitation of the first fluorophore will cause excitation of the second fluorophore when the two are in close proximity. The proximity-dependent excitation of the one of the fluorophore is one variation of FRET. FRET may be used to provide highly sensitive detection of binding (and therefore concentration).

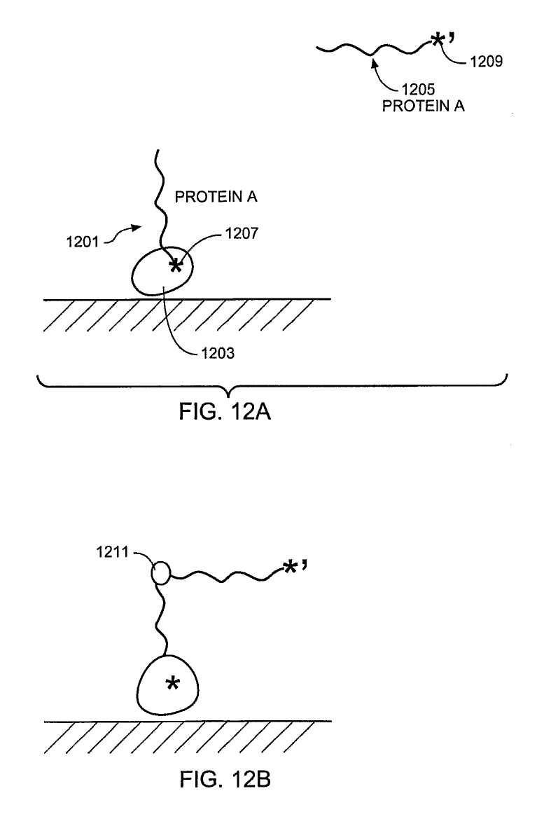

FIGS. 12A and 12B show one example of using FRET as part of a product sensor. In FIG. 12A, Protein A acts as a bound capture element 1201 as described above, because it can bind to the product being produced in the well/bioreactor. The capture element may be labeled directly, or it may be bound to a florescent bead, such as a florescent latex bead 1203, as shown. For example, a florescent latex bead may have a diameter of between about 0.1 to 20 μm. In general, the florescent bead (or otherwise fluorescently labeled capture element such as an antibody or target binding partner) is labeled with a fluorophore 1207 that has an excitation wavelength of “X” μm and an emission wavelength of “Y” μm. In this example, free Protein A is also present in the solution, and is labeled with a second fluorophore 1209 which has an excitation wavelength of “Y” μm and an emission wavelength of “Z” μm. In some variations, a second or different capture element is used as the free (unbound) capture element, preferably a capture element binding to a different region of the product. Unlabeled product is released into the well, where the product sensor may detect and measure it. In operation, when the product 1211 is released into the well it may be bound by both the free 1205 and the tethered 1201 capture agents, bringing the two different fluorophores 1207, 1209 into proximity so that when the sensor is excited with a wavelength of “X” μm, the signal at wavelength “Z” μm indicates that the product is bound and FRET will occur, as shown in FIG. 12B. The florescence intensity may be proportional to the amount of product in the well.

In one variation, the product may be labeled as it is produced, for example, by incorporating florescent regions (e.g., GFP, etc.). Any appropriate florescent label may be used, or appropriate pairs of florescent labels. For example, the labels 1207, 1209 maybe, e.g., dyes such as Rhodamine, FITC, cy3ₚ cy5, etc., dye proteins such as GFP, PE, etc., latex beads, quantum dots of any available size and color, gold nano particles, etc. The florescent colors utilized may include, but are not limited to ultraviolet, visible, infrared, near infrared, etc.

In one example, monoclonal IgG may be synthesized from hybridoma cells in culture using a FRET based immunoassay. Hybridoma cells may be cultured in suspension at 37° C. and 5% CO₂ with necessary nutrient feed, as described elsewhere herein. The hybridoma cells may produce IgG molecules in solution. Synthesis of IgG may be monitored as it is released into the well using the product sensor described above. Thus, Protein A can be covalently coupled to a 6.7 μm microsphere that emits florescent light at ABC nm when excited by light at XYZ nm. These Protein A coupled beads may be held in a porous patch that can be interrogated by a source of XYZ nm light with quantification of ABC nm emission. As IgG is synthesized and secreted into the medium by the cultured hybridomas the IgG molecule will be labelled on their Fc portions with quantum dots coupled to Protein A. The ‘Protein A quantum dot IgG” complex is free in solution, and can also bind the Protein A coupled latex beads in the porous patch, as described above. As these complexes are bind and are brought into close proximity with the latex beads and the fluorophore they contain, binding will be quantified via a FRET event that generates photons as a consequence the Protein A quantum dot IgG complex binding event. The extent of the FRET event may be proportional to the concentration of the IgG present in solution and will be used to directly quantify, from calibration data, the number of molecules of IgG present in solution.

Sensors may be connected to a control unit which may use the sensor information to regulate one or more operation of the smart plate. For example sensors may monitor the level of media within the wells (e.g., an optical detector may detect the meniscus of media within the well), and may output to a controller. The controller may include control logic for regulating the amount of fluid within the well based on (at least) the output of the optical sensor. Thus, if the level gets too low, the controller may cause the valves connecting the ports of the wells to the medium source, and allow more medium to be provided. Once medium gets too low, the entire process may repeat itself. Similarly, if the well gets too full, a drain port may be used to remove excess medium.

The control unit may also record or further analyze data from the sensor. The control unit may be connected to the plate by an electrical lead (e.g., a wire) or by a wireless connection. In some variations, the same control unit may be used to control a plurality of plates. In some variations, a control unit may be on-board the plate. The control unit may comprise software (e.g., analysis or control decision logic).

The control unit may also be programmable. For example, the control unit may permit user-entered temperature control, flow rates (e.g., flow of material through the ports), wash cycles (again controlling flow of fluid in and out of the ports), and temperature/feeding/treatment cycles. The control unit may also integrate with any other portion of the system, including an imaging unit (e.g., microscope), to control the applied light (e.g., white-light, UV-light, etc.) for time-lapse or extended recording periods.

The control unit may receive user input. For example, the control unit may interface with a computer (or may be part of a computer). The control unit may communicate with commercially available software such as LabView™ (National Instruments). In some variations, the controller may include separate controller logic. The controller may regulate, for example, the pH of the solution (e.g., by monitoring the pH directly and responding, or by controlling the CO₂/O₂ mixture), the addition of drug (e.g., experimental or treatment drug), the addition of new media, the removal of media, or the removal of sample (e.g. aspiration of material from the cell culture). Although a single multi-purpose regulator may be used to control (and or store and analyze) all or any of the features described herein, separate (e.g., dedicated) controller units may also be used.

Any appropriate control unit may be used with any of the plates described herein, including plates having any of the sensors (or none of the sensors). For example, in one variation, a control unit controls only the temperature of the plate. Thus, the control unit may include one or more connectors (e.g., plugs) for connecting to the electrodes that heat the plate (and/or the lid of the plate). The control unit may also connect to the temperature sensor or sensors. In some variations, the control unit includes a display for indicating the temperature (actual or relative temperature) of the plate or regions of the plate (e.g., individual wells or the lid). In one variation, the lid and the base of the plate include separate temperature controls, and each region (the lid and the base) include a separate temperature sensor. The temperature control (e.g., connecting to the electrodes) and the temperature detector both input into the controller. An operator may monitor and adjust the temperature using buttons, dials, switches, etc. on the controller.

In one variation, the controller controls both the temperature and the application of a gas mixture (e.g., 5% CO₂) to the plate. Thus, the controller receives input from the user to adjust the temperature (e.g., the temperature of the bottom of the plate and/or the lid), as well as the rate that a gas mixture is applied. The rate of the gas mixture may be adjusted using the controller. A controller may include a meter or display indicating the current gas flow/pressure, and the user may adjust it either manually (monitoring the effect on the current gas flow) or automatically. For example, a target (or target range) of gas flow may be selected by the user, and the controller may automatically adjust the gas flow to be within the target range. Thus, the controller may include a detector for detecting the gas flow, as well as an indicator. The temperature(s) may also be similarly controlled. The controller typically includes control logic for controlling the gas flow and/or temperature.

In operation, each well may include one or more sensor, as described. These sensors may be used to detect multiple different parameters. Each sensor may be individually activated (e.g., excited), and the signal or response detected, and the response analyzed. The steps of activation, sensing, detection, and analysis may be performed separately for each sensor, or they may be combined. For example, multiple sensors may be activated by a single activation, and multiple sensors may be detected by a single detector. Any or all of the sensors described herein may be included or may be incorporated as part of each well.

Each sensor may be activated, detected and analyzed independently. For example a sensor in a well (or a region of a well) may be activated (e.g., by emitting light of a particular wavelength) to excites a first optical sensor (e.g., a pO₂ sensor). The response of the sensor (e.g., absorption and/or emission) is then detected by a detector, and the detected response from the sensor is analyzed. The response typically reflects the characteristic of the system or a parameter of the culture conditions within the well (e.g., pH, pO₂, pCO₂, etc.). A second round of activation/sensing/detection/analysis may then be performed on the same or a different sensor.

In multi-well systems, a sensor or sensor may be activated/detected/analyzed in each well selectively, simultaneously (e.g., in parallel), sequentially, or some combination thereof. For example, all of the pH sensors in a plate of multiple wells (e.g., multiple micro-bioreactors) may be activated, detected, and analyzed at the same time, before another sensor (e.g., pO₂ sensor) in all of the wells is activated, detected and analyzed. In some variations, multiple sensors are activated, sensed, detected and analyzed at the same time in a single well.

The plates described herein may also include mixers (e.g., magnetic mixers) for agitating material (e.g., medium) within the wells.

### Mixers

In general, an entire plate may be mixed or agitated by moving the entire plate. For example, each well of an entire smart plate may be mixed by placing it on a rocker or shaker. However, it may be desired to mix only some of the wells, or to mix material within the wells while the rest of the plate remains stationary (allowing visualization, etc.). Thus, individual mixing may be achieved within each well of the plate by including one or more integrated stirrers operably connect to one or more of the wells. For example, an integrated stirrer may be an integrated magnetic stirrer having a multi-pole magnetic source that at least partially surrounds a well of the tissue culture chamber.

In one variation, mixing of liquid within the wells (particularly small wells, e.g., less than 10 ml volume) may be performed using paramagnetic beads for non-contact mixing. These mixers may also be referred to as stirrers. Thus, the plate may include one or more magnets configured to move the magnetic beads (or some other magnetically responsive stirrers) within the media to agitate the media. By alternating the magnetic force applied to paramagnetic beads within the media of a well, the media can be mixed as the beads are moved back and forth within the well by the action of the magnetic forces applied.

Magnetic force F on a magnetic bead of mass, m, creates an acceleration, “a” and this force F=ma force the bead to travel S, the well diameter. With initial velocity, u, being at rest at zero(0), S=(½)at². For a 6-well plate, the well diameter S can be approximately 35 mm (e.g., for a 96-, 384-, or 1532-well plate, Sₘₐₓ is approximately 9.5 mm, 4.25 mm, or 2.125 mm, etc.).

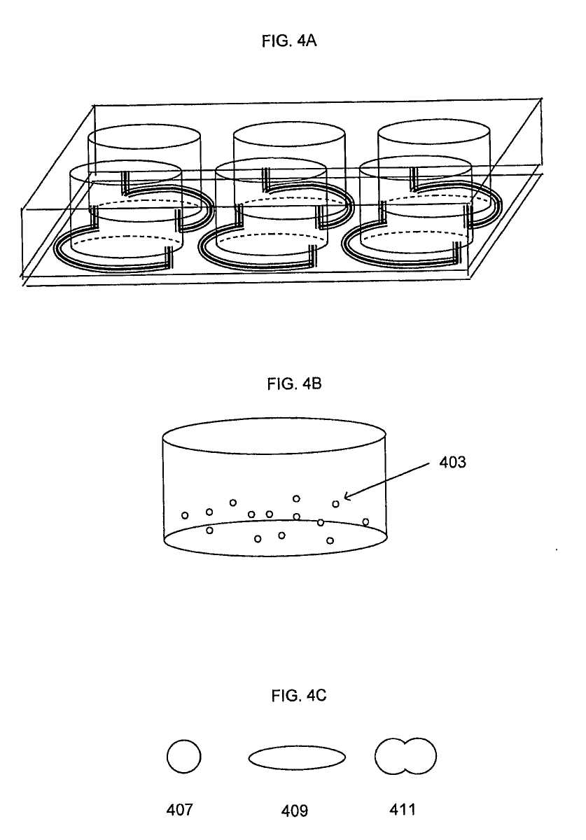

FIG. 4A shows one variation of a smart plate including a controllable magnetic stirrer as described. In FIG. 4A, the plate includes a multi-pole magnetic ring extending at least partly around the well, as shown. The acceleration “a” can be determined for a given medium in this arrangement (e.g., based on the viscosity, temperature etc. of the medium). Using F=ma, acceleration, “a” can be derived for various F values. Thus, there will be a minimum value of force, F, for which the bead reaches the opposite magnetic pole. Any force above this will certainly give higher acceleration. Thus, the activation of the magnet can be determined by noting the time that a bead may take to travel, t. The polarity on electromagnet may thus be changed to keep force F constant. This makes bead travel in the opposite direction making one complete cycle. Based on desired frequency, the force F is changed up or down based on desired cycles. Using this needed frequency (or a calibrated cycle time), a well's micro electromagnetic polarity may be changed electronically (e.g., by the control unit connected to the source of the magnetic field (e.g., the multi-pole magnetic ring shown in FIG. 4A).

Electromagnets (providing the force to move the beads) may be provided in any appropriate position, typically outside of the well (e.g., the top, sides, bottom of the wells, the lid, etc.), and in any appropriate arrangement. In some variations, the electromagnetic comprises a multi-pole magnetic ring around each well. In some variations, the multi-pole magnetic source may form a portion of the well body.

FIG. 4B shows a well including magnetic or paramagnetic beads 403 that are acted on the by the multi-pole magnetic source, as described above. FIG. 4C shows different variations of stifling beads, including round 407, barbell 409, and oblong 411. The shape of the beads may be optimized to increase the surface area, thereby increasing drag volume which (in return) will increase mixing efficiency. For example, round beads may be used, or beads having cavities, projections (e.g., “arms”) propeller-shapes, concave surfaces, or the like. The shapes may be limited to fluidic height and may be small enough to minimize blockage of the optical path when at rest, allowing imaging or image based cell counting time. In some variations, during imaging, or image based cell counting, the beads may be rested at any one of the poles. Shapes of different sizes and shapes may be used (e.g., a range of shapes and sizes) at the same time. For example, multiple sized paramagnetic beads, of various shapes may be used with an electromagnetic ring located outside of the optical path of the plate. Thus, the beads may comprise a magnetic or paramagnetic material.

Beads may also be sterile or sterilizable to prevent interference with cell culture embodiments. In some variations, the beads may be coated with a protective layer (e.g., a biologically inert layer, such as a latex or other plastic).

In some variations, a single bead (or stirrer) is used, or multiple stirrers may b used. The stirrer may correspond to a magnetic or paramagnetic shaped bead. When a single stirrer is used, it may be shaped so that the electromagnetic rotation causes substantial displacement of media within a well. For example, a single propeller may be propeller-shaped or have one or more surfaces configured to push fluid. Any appropriate shape may be used. The stirrer may be configured to float within the medium of the well, or on top of the well. In some variations, the stirrers are configured to rest on the bottom of the well. The stirrers may be small and light enough so that they are suspended within media of the well, so as not to interfere with adherent cells. Thus, the electromagnets acting to move the stirrer may be positioned above the bottom of the well (e.g., slightly above the bottom of the well).

In one example of the operation of a magnetic mixer, mixing within a well of the plate may be performed by placing the beads within the well containing a fluid (e.g., culture medium), and turning on the magnetic field to cause the creation of a magnetic pole (“A”) at one or more parts of the electromagnet source. The magnetic pole “A” may be maintained in a stable position for a predetermined amount of time (e.g., “t” or a fraction of “t”). In some variations, the magnetic pole “A” may move in a continuous fashion. After the predetermined amount of time has passed, the magnetic pole “A” is turned off, and a new magnetic pole “B” (typically located on an opposite end of the well) is turned on for the predetermined time “t” (or a fraction of “t”). These steps of turning on and off poles “A” and “B” may be repeated, causing the magnetic or paramagnetic beads to move within the well. Any type of motion (e.g., around the perimeter of the well, across the well, up or down, etc.) may be directed by controlling the electromagnetic source, guiding the beads. The stirrer beads may be resident in the well (e.g., they may be included as part of the well prior to adding media, samples, etc.) or they may be introduced thereafter. For example, beads may be introduced through one or more ports of the well.

Thus, the mixing system described herein may be used as part of a smart plate as described herein (e.g., as part of a micro-titer plates, microarray, micro-, mini- or large-bioreactor built in plastic, glass, silicon or any non-magnetic materials).

Software or magnetic stirring control logic may also be used to control the stirring of the magnetic stirring described.

### Systems

As described above, the smart plates (or slides) described herein may be used as part of a system. Systems including the multi-well plates described herein may include systems for monitoring, imaging, culturing, labeling, or the like.

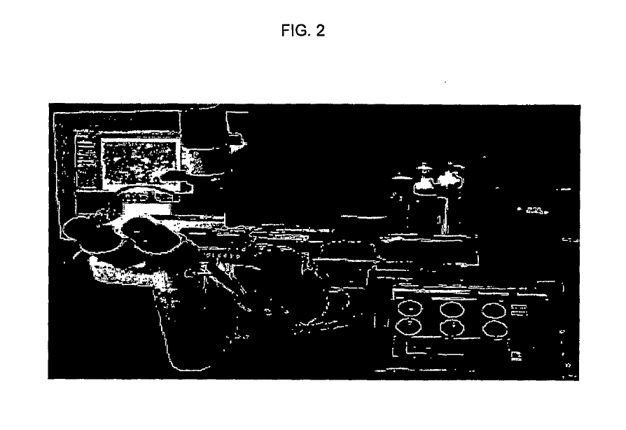

For example, FIG. 2 shows one variation of a system for culturing and monitoring live cells including a controllable plate as described herein. The plate used with this system may include any of the features (e.g., sensors, temperature control, flow control, etc.) described. For example, in FIG. 2, the plate is used with a real-time imaging platform based. The smart plate conforms to a standard 6-well micro titer plate format, and includes temperature control and regulation of gases such as O₂/CO₂/N₂ (and regulation of pH). This system allows researchers to take time-lapsed images of adherent cells over an extended period, while the plate is on an inverted microscope, without requiring that the plate be taken in and out of the incubator both in perfusion and static nutrient flow conditions. The plate is connected to a control unit which regulates the flow of media, the temperature of the plate, and may interface with the microscope, as shown. A computer may be used to interact with the microscope (e.g., storing digital images, etc.) and/or the control unit for the smart plate.

The system shown in FIG. 2 includes three primary components: a disposable smart plate (cell culture slide), a controller (e.g., controller unit), and the control software (e.g., “smartware” software controlling the smart plate). The bottom layer of the multi-well plate (slide) in this example is coated with a conductive thin film layer (e.g., ITO) to provide stable heating of cell culture walls. A controller connects to a computer and using control software, manages the temperature of the slide as well as CO₂ and nutrient flow at programmed levels to approximate the natural host environment of the living cell. This system may integrate into existing imaging platforms and may be adaptable to individual system needs. For example, when using an inverted microscope with an oil immersion objective, the system may include a simple but effective heater for the objective lens.

Thus, this system may make it possible to investigate, in real-time, biological questions that are either temperature and/or time dependent by approximating an in-vivo environment under the microscope.

### Potential Uses

The controllable multi-well plates described herein are not limited for cell culture or imaging uses. As described above, the smart plates may be used in any situation where it is desirable to provide a plate having one or more wells (especially small wells or wells having small volume), where the plates include on-board control of any of the following features: temperature controlled; fluid flow (into or out of the well) control; monitoring (e.g., optically or by any other appropriate sensor) of conditions within the well environment; and/or stirring of material (e.g., liquid) within the well.

Example of applications which may benefit from these plates include, but are not limited to cell culture applications (e.g., culture of bacteria, tissues, etc.), growth or culture of antibodies (e.g., monoclonal, polyclonal, etc.), purification of antibodies, miniature bioreactors (e.g., fermentation, production of proteins, etc.), purification of water, testing for pathogens (in air or water), testing or screening of drugs (including bioreactivity and bioabsorption of drugs in different tissues), cell labeling or staining (including immunihistochemical applications), etc.

For example, one benefit of the smart plates described herein is that the array of wells that form a single controlled plate may be linked together. In one variation, the multi-well smart plates can include cultures of tissue types from different portions of the body (intestinal, vascular, liver, neuronal, etc.). The effect of identical concentrations of a drug on all of these different tissue types may be assessed by adding drug to them all simultaneously. In addition, the effect of any drug byproducts may be assayed by adding drug first to the well including the tissue that would see the drug first when taken by an intact subject (e.g., the stomach/intestinal tissue), and then transferring media from this well into wells containing tissue cultures from downstream tissues. This novel assay may therefore allow the investigation of the effect of bioabsorption of the drug in different tissue types, as well as the effect of any potential metabolic byproducts of the drug which may result from one or more tissue type.

In another example, multi-well smart plates may be used as part of a small-scale multi-fermentor system. Thus, each well of the multi-well plate may individually regulate a micro-environment for the growth of cells (e.g., bacteria). Since the wells may be isolated from each other, many parallel fermentation processes can be run simultaneously, with little risk of cross-contamination. For example, when engineered bacteria are grown, samples of the bacterial culture may be removed (e.g., from one of the ports, including a filtered port) and tested. Bacterial growth may be optimized and controlled.

### Making or Manufacturing Multi-Well Plates

In general, a multi-well plate as described herein may be produced or manufactured in any appropriate way, including blowing, molding, and CNC manufacturing. In some variations, the multi-well plates described herein may be manufactured by injection molding. For example, individual component parts of the multi-well plates may be injection molded (en masse or individually) and assembled. Injection molding is typically inexpensive and accurate, and may be allow a wide range of materials (e.g., polymeric materials) to choose from.

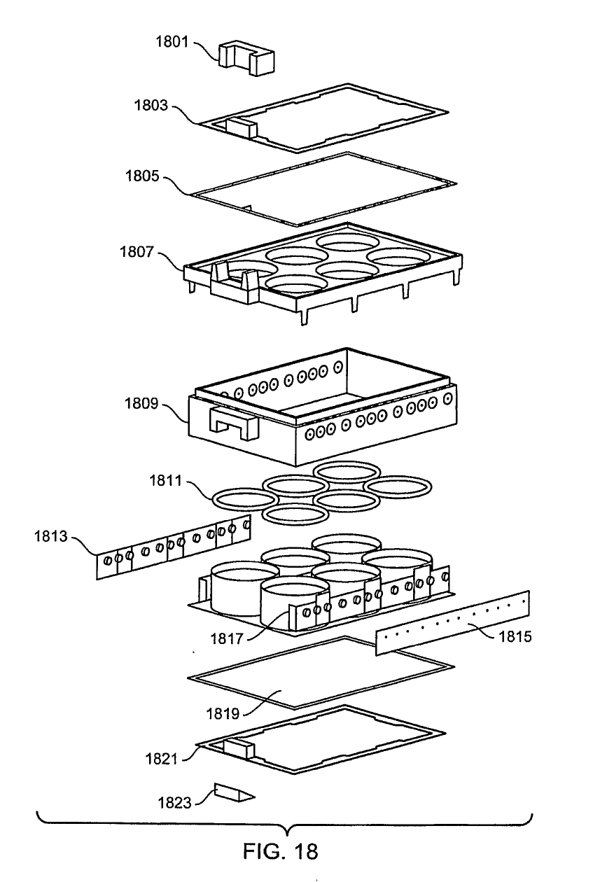

FIG. 18 shows an example of an exploded view of all of the parts of a single multi-well slide, as described herein. The individual components may be assembled (manually or by an automated process). In FIG. 18, the lid includes a removable RTD (temperature sensor) assembly 1801 (e.g., similar to the mount shown in FIG. 17), that engages the molded lid 1807. An ITO coated heater glass 1805 is attached to the molded lid 1807, and a lid flex circuit 1803 forms an electrical contact with the electrodes on the ITO coated glass 1805. The lid may engage the outer shell 1809 of the plate using snap clips which may be incorporated into the molded lid. The lower half of the plate is assembled by attaching or overmolding left 1813 and right 1815 septum material (e.g., formed from a relatively soft elastomeric material) into the inner shell 1817, placing the seals (o-rings) 1811 in contact with the inner shell 1817, and then mounting the lower heater 1819 (a glass plate coated with ITO) with attached flex circuit 1821 and plate RTD assembly 1823 when snap clamps are used a corresponding receptacle must be provided for each clamp in the outer shell.

Manufacture of a multi-well slide as described in FIG. 18 may include the following steps: forming individual parts; overmolding the inner shell 1817 with the left and right sides 1813, 1815 to form a septum; inserting the inner shell 1817 with attached seals 1811 into the outer shell 1809 to form the plate assembly; installing the heater assembly (coated plate 1819, attached electrodes 1821 and RTD assembly) into the plate assembly. The lid is a separate assembly which consists of heater assembly (coated glass 1805 and attached electrodes 1803 and RTD assembly) into the lid frame 1807. The finished lid assembly may be installed by the customer after the live cells and media are inserted into the plate.

Individual parts may be formed by any appropriate method. Some parts may be injection molded. For example, the outer shell 1809, inner shell 1817, lid frame 1805, and lid and plate RTD assemblies may be injection molded. In some variations, the plate (or well) flex circuit may be constructed as known in the art. For example, the flex circuit may be formed of layers of polyamide and copper. Positive and negative contact layers (e.g. a positive and a negative plane) may be layered so that contacts can be exposed to connect to the conductive layer (e.g., ITO). A stiffener may be used to stiffen the flex circuit, and connectors (e.g., connecting the flex circuit to wire and thus a power supply) may be soldered on.

In some variations, the well heater assembly is assembled by first cleaning the ITO coated class, then silk screening the electrodes with conductive epoxy and flash curing them. Silver epoxy may then be applied to the contacts of the flex circuit, and the entire assembly may be cured at 125° C. and baked flat until cured. The same general process may be used to form the top heater assembly (micro-heater) as well.

A smart slide plate (or well) assembly may be built by pressing the inner shell 1817 into the outer shell 1809. The inner and outer shell may be friction fit, or may include mechanical engagements to secure the two together. In some variations, an adhesive may be used to secure the inner and outer shells together. As mentioned previously, the inner shell and the outer shell may create one or more air gaps between them, which help thermally isolate the wells of the plate from the surrounding temperature. For example, an air gap may separate the bottom of the well (e.g., the glass bottom) from the surface on which the plate rests (e.g., microscope stage). The air gap may extend around the individual wells, or one or more separate air gap(s) may be formed around them. After connecting the inner and outer shells, the heater assembly (the plate heater assembly) may be connected. For example, the plate heater assembly may be glued to the well assembly using silicone adhesive. A connectors (e.g., a flex cable) providing power the flex circuit to heat the plate may be positioned within the inner shell before installing the coated heater assembly. The lid assembly (including a lid heating assembly) may be assembled in the same fashion.

In some variations, a seal such as an o-ring seal is included between the lid and the well assembly. Thus either (or both) the lid or the well assembly may include a seat or contact for holding a seal. For example, an elastomeric (e.g., rubber, silicone, etc.) o-ring seal may be inserted around an inner perimeter of the inner shell of each well of a plate. When the lid is closed over the plate, a lower edge of the lid engages the o-ring, forming a seal.

Although a simple method of making and assembling a multi-well plate is described above, it should be understood that many variations of these manufacturing and assembly techniques are possible, including other methods of making and assembling the individual parts, or of combining individual parts. Furthermore, some assembly steps may be included, such as coating the ITO (or other transparent conductive material) with a protective coating, and attaching the temperature sensor(s), or additional sensors.

Described below are examples of different multi-well (e.g., “smart”) plates as described herein, as well as different applications of these plates.

### Examples

One variation of a multi-well smart plate (or slide) is described below. In this example, a multi-well slide comprises a 6-well slide that is SBS compliant, having dimensions 5.0″×3.3″. This plate is connected to a fluid controller (that has dimensions 12.00″L×12.00″W×3.5″ H) and an electronic controller (having dimensions 5.5″ W×12.00″D×12.00″H). The plate also includes a micro-heater as described above, capable of regulating the temperature between about 30° C.-50° C.±0.10° C. The plate includes a heated lid. The heated lid also regulates temperature between about 30° C.-50° C.±0.10° C. The system may also include a microscope objective lens heater that controls the temperature of the objective (e.g., oil or water) over the same range of temperatures (30° C.-50° C.±0.10° C. ). A power supply (e.g., 12V/10 W) is readily attachable to the plate (e.g., base and lid, or objective heater) for powering the heater(s). Finally, where gasses like CO₂ will be supplied to the plate, the system may include a CO₂ humidifier bottle heater that regulates the temperature of the supplied gas over the temperature range of 30° C.-50° C.±0.10° C.

This system (e.g. the sensors on the plate, or the controller) may also include one or more alarms for signaling when any of the environmental parameters are outside of a preset range. For example, when the CO2, temperature, well volume flow rates, etc. exceeds some allowable range (or optimal range for cell growth). The flow control (e.g., though the port into or out of the wells) may be regulated at a rate of approximately 0.0452 mL/sec. Flow from the drain may proceed at approximately 0.052 mL/sec.

This example is provided to illustrate some of the components (and their practical ranges) for one variation of a system including a multi-well smart plate, as described. Other variations of the plates and system incorporating them are possible.

### Example 2

### Proof of Concept

A proof-of-concept experiment was done comparing an incubator using a standard (e.g., passive) multi-well plate with a smart multi-well plates as described herein, in which temperature and CO₂/O₂ were regulated on-plate. Researchers at University of Southern California in the Keck School of Medicine use the development of chicken skin appendages as a model for organogenesis (see references 1 and 2). Embryonic skin explants are monitored for the appearance of dermal condensations seen as dark circles on the skin. These researchers study the signal process between the dermis and the epithelium that leads to the fundamental organization of periodic patterning for feather development. In a recent experiment, one variation of a 6-well smart slide was used to monitor dermal condensations in chicken skin cultures.

Materials and Methods: Embryonic day 6.5 skin explants were grown in HEPES buffer (10 uM) with Dulbecco's Modified Eagle's Medium (DMEM) supplemented with 10% fetal bovine serum plus gentamycin (diluted 1:1000). Tissues were placed into the bottom of Cell Culture Inserts with 0.4 u membrane bottoms (BD #35 3039) and placed into either standard 6-well Falcon tissue culture plates or the 6-well smart slide. Cultures were grown for 17 hours in either a humidified incubator at 37 deg C. (Falcon plates) or in the free standing smart slide. Time-lapse video images of the explants in the smart slides were taken at 15-minute intervals in order to assess the development and overall health of the tissues.

Explants in the Falcon plates were imaged at the start and at the end of the 17-hour experiment. The smart slide 6-well culture well plate was programmed through a controller unit and a laptop running software to provide the following conditions for the experiment: smart slide temperature 37.0 deg C.; smart slide cover 37.5 deg C. (to eliminate condensation on the top cover); gas flow rate 10.0 sccm; nutrient flow to each well 2.0 mL/well; drain well volume 3.0 mL/well; well drain-fill cycle every 300 minutes.

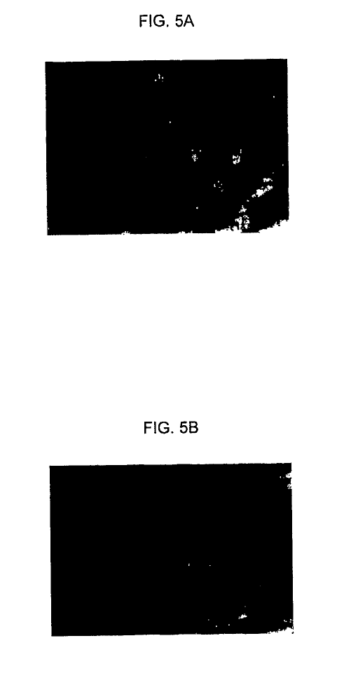

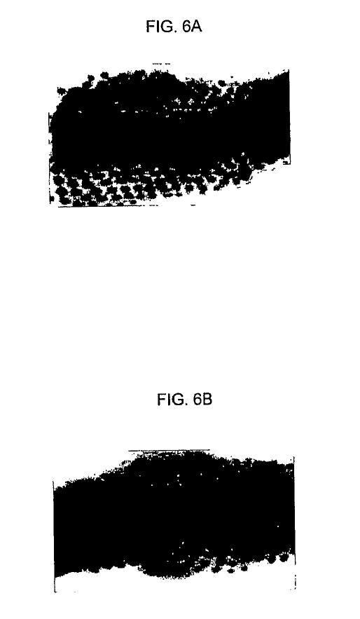

FIGS. 5A and 5B show the feather bud formation at 0 time (FIG. 5A) and at 17 hours (FIG. 5B) in the Falcon plate. FIGS. 6A and 6B show a side-by-side comparison on explants grown in the incubator (control 6A) and the smart slides (6B).

Results: Developmental progression of feather bud growth from explants grown in Flacon plates in an incubator and in the stand-alone smart slide were then compared after the 17-hour experiment. (FIGS. 6A and 6B). The researchers stated that, “Survival and growth appeared to be similar in explant cultures grown in our incubator and the smart slide chamber.

Thus, the smart slide is an ideal tool for long-term cell imaging; and cell culture growth when long term optical or metabolic monitoring is required. Experimental conditions may be entered into computer software for controlling temperature, gas and nutrient flow during the duration of the experiment. Wells may be filled and emptied at times selected by the user or run into the wells in a continuous mode. Nutrient and gas into each well are independently controlled. The 170μ thin optically clear well bottom (in this example) allows both light and fluorescent microscopic imaging. An objective heater and sensor may be used to fit onto any standard lens for oil immersion work. Temperature and liquid flow rates may be recorded for the duration of the experiment (minutes or days) and provide a complete printed record for documentation. Thus, long-term imaging experiments may be performed in an operator independent mode.

Thus, this system may allow advantages such as: study of live adherent cells for long term imaging; 6-well formatted, micro-incubator-in-microtiter-wells; adherent cells, tissues, infected cells, etc. can all be handled with no cross contamination; real-time nutrient flow, temperature, CO2 may be tracked by well; inverted microscopes may be used to monitor development.

References: (1) Jiang, T-X., et al. Self-organization of periodic patterns by dissociated feather mesenchymal cells and the regulation of size, number and spacing of primordia. Development 128:4997-5009 (1999). (2) Yu, M., et al. The developmental biology of feather follicles. Int. J. Dev. Biol. 48:181-191 (2004).

### Example 3

### Smart Plate

FIG. 3 shows another variation of a smart multi-well plate in which a number of sensors are included. In FIG. 3, the plate 1 is shown as a six-well plate, wherein each well 2 is an inner diameter of 35 mm and a depth of 19 mm. A single well is shown schematically in cross-section (indicated by the arrow 2). This well includes a cover and four ports passing through the wall for applying or withdrawing fluids (e.g., media, gasses, drugs) to or from the well. For example, a NaOH μ-pump (which may operate at 160 nL/pulse) may be connected to one port, which is controlled by a valve 5. An gas supply (e.g., O₂ supply) may be connected to a second port 6. Media my be applied through a feeder port 7, and samples of media (or excess media) may be removed through the sample port 8.

Each well of the example shown in FIG. 3 may also include sensors for detecting characteristics of the environment within the well. For example, the well includes a pH Sensor 3, and a dissolved O₂ sensor. These sensors comprise an emitter and a detector that pass light through the well (since the bottom and top are at least partially transparent), and detect pH or dissolve O₂ based on the absorption through the media within the well. Non-contact sensors such as these are commercially available from, e.g., Fluorometrix™). Of course, other sensors may be used, including sensors within the well itself.

The well of FIG. 3 also includes a cell counter 11 for optically counting cells within the well (e.g., based on optical density, a ratiometric determination of cell density). The sensors (and/or a temperature control, as well as the control of the valves and ports described above) may all be controlled and monitored by a controller 9, which may be connected to a computer. As previously described systems including multi-well plates may include additional components such as imaging systems, microscopes, output devices (printers, etc.), storage devices (e.g., memory, disks, RAM, etc.), control software, analysis software, or the like.

This invention has been described and specific examples of the invention have been portrayed. While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well. Finally, all publications and patent applications cited in this specification are herein incorporated by reference in their entirety as if each individual publication or patent application were specifically and individually put forth herein.

### Example 4

### Micro-Bioreactor

The devices described herein may also be configured as a micro-bioreactor (or micro-fermenters). A single well, or multiple wells including any (or all) of the features described above may be configured for complete optimization of a bioprocess, including production of cells, or production of cell byproducts from cells. Such “micro” bioreactors may be used to determine or optimize growth and culture conditions, and may be used to guide optimization before scaling up to a large (e.g., 1000 L or greater) batch size, beginning with ml size batches. In the past, reactor optimization was commonly done with 1000 mL flasks, leading to waste and expense.

When the plates are used as micro bioreactors, many (or all) of the components may be incorporated into a small-sized plate into a comprehensive design system. For example, the plates may include control of temperature, agitation (stiffing) addition of gasses and gas mixtures (e.g., O₂, CO₂, N₂, etc.), detection of product, detection of pH, etc.

A micro-bioreactor may include any of the following features: multiple wells, cell imaging, temperature control, media control, gas exchange, media adjustment, disposability, reactor well mixing, cell number counting, pH monitoring and/or adjustment, pCO₂ monitoring and/or adjustment, pO₂ monitoring and/or adjustment, and product monitoring.

Multiple micro-wells may be used as part of a micro bioreactor. For example, a six-well slide described above may be used (or 12-well, 24-well, etc. sizes). The multi-well plate may eliminate the problems and waste associated with performing cell growth optimization in individual 500 mL or 1000 mL flasks.

The micro-bioreactor may also be configured to allow imaging of cells from any of the micro-bioreactor wells. For example, the bottom of the wells may be configured of 175 μm-thick glass, and the cover of the well may also be configured of glass or a sufficiently transparent material to allow illumination and imaging of the cells without having to remove a cell sample (as is required when growing cells in flasks). For example, the micro-bioreactor may be used with an inverted or upright microscope, as previously described.

The temperature of the plates may be regulated. The wells (individually or as a group) of a plate may be temperature-controlled to within 0.1° C. across the wells at user-defined temperatures. Similarly the tops (or covers) of the wells may also be regulated. Regulation of the tops of the wells may also help prevent condensation on the top, enhancing imaging an signal detection through the plates. This temperature regulation may eliminate the need for incubators and heating mantles, as is required with flasks.

Each well of a multi-bioreactor (e.g., plate) typically includes fluidic channels that allow introduction of media at user-defined frequencies, volumes, rates (e.g., μL/min), etc. Cells may remain in the wells (e.g., when adherent), or may be prevented from leaving by filtration, etc. Small media volumes (e.g., between 1-3 ml per well) may be used. The wells may also be calibrated to indicate approximate volume. In addition to fluidic channels, each well a multi-bioreactor may also include in/out lines for the introduction of pre-mixed gasses at user defined rates and frequencies. For example, up to three different gasses (e.g., N₂, O₂, CO₂) can be mixed at user-defined ratios, and applied to the mini-bioreactor.

One or more of the inlet lines into the wells of the micro-bioreactor may be configured to introduce media adjustments (e.g., buffers) into the well or wells. For example, dilute sodium hydroxide may be added to adjust pH. Media adjustment may be user determined as to rate of flow, frequency, etc. In some variations, the media adjustment may be automatically regulated. For example, the rate of flow and frequency of adjustment may be determined based on feedback control (e.g., from the controller).

A plate configured as a mini-bioreactor may also be disposable, or configured for single-use. Thus, the mini-bioreactor may be used for a predetermined time period (e.g., hours, days, weeks, months) before being disposed (or recycled). In some variations, the micro-bioreactor may be reused (e.g., by sterilizing it, and/or reconditioning it).

A micro-bioreactor may also include individual well mixing, as described above. Each well may contain magnetic mixers (e.g., the magnetic mixing beads). This magnetic mixing may allow the user to select mixing speed and frequency (or it may be automatically selected), eliminating the need for rockers or separate mixers. Mixing can be halted on demand, (e.g., when imaging).

The micro-bioreactor may also include a cell counter. The micro-bioreactor may include one or more integrated counters (or portions of a counter, as described above), or it may include a window region to allow cell counting by an external cell counter. For example, the micro-bioreactor may be adapted to connect to a cell counter that scans each well of the micro-bioreactor (e.g., multi-well plate) or that simultaneously counts cells from all (or a subset) of wells of the micro-bioreactor(s). Cell counting may be performed in real time. Each well may be monitored for cell growth using (for example) the turbometric cell counter described above. Cell counting may be preformed without contacting (e.g., without removing a sample of) the bioreactor. Cell counting may be automated. For example, the controller may automatically (at a user defined interval) count the number of cells.

Micro-bioreactors may also be monitored and regulated based on any appropriate sensors, including those described above. For example, a pH sensor may monitor the pH in each well. In one variation, a pH sensor is a non-contact fluorescent patch sensor for pH. The gas concentration may also be monitored, including the pCO₂ and pO₂. Non-contact pCO₂ and pO₂ florescent patch sensors may be used, for example. Readings from any of these sensors may be used to modify the bioreactor environment. For example, the pCO₂ and pO₂ levels may be feed back to regulate the applied gas mixture (e.g., the % CO₂/O₂/N₂ mixture). Similarly, the pH sensor may feed back to regulate the pH of the chamber by adding acid/base mixtures or otherwise modifying the pH of the system.

In addition to monitoring and regulating the environment of the micro-bioreactor, one or more sensors (or product monitors) may be included for monitoring the expression of a product, particularly products that are released into the media by the cells growing in the micro-bioreactor. The product monitor may be a patch immunoassay detector, as described above.

The micro-bioreactor may be controlled by one or more controllers that can regulate and coordinate the activity of the sensors and active control features (e.g., temperature, stirring, media adjustment, etc.). A controller may include controlling logic for controlling individually features (e.g., temperature, sensors, stirrers, etc.), or for coordinating multiple features. For example, the controller may include logic configured to receive sensor information on pH, cell count, gas concentrations (pCO2, pO2, etc.), product concentration, temperature, and the like, and for using this information to adjust the environment of the micro-bioreactor (e.g., temperature, media adjustments, gas mixture added, sample extraction, etc. The controller may also include user inputs, and may provide output. Output from the controller may be sent to a display (e.g., LED, monitor, printer, etc.), an alarm (e.g., a bell, chime, light, etc.), a memory (e.g., digital memory, memory media, etc.), or an addition computer system.

The controller and the controller logic may include both software and hardware. In some variations, the controller logic includes feed-back control. The logic may include feedback control thorough decision trees and algorithms which may regulate the frequency, and manner in which the micro-bioreactor is monitored and adjusted. For example, the controller may automatically select and adjust individual wells. The controller may assist or automatically control cell imaging (e.g., taking, storing, and analyzing images of cells at certain locates in the well, at certain frequencies, etc.). The controller may regulate the temperature (both lid and the base of the plate or wells), as well as the media control (e.g., adding new media). The controller may also sample the gas, and adjust the gas exchange and mixtures (ratios) based on pre-set values, or based on sensor output. Similarly, the media may be adjusted (e.g., pH may be adjusted by addition of NaOH, etc.), at predetermined or automatically determined intervals. The cells and media may be mixed (stirred) continuously or at some preset or automatically determined intervals (e.g., using the magnetic stirrers described herein). The cells within a well may be counted at set or automatically determined intervals. pO₂ and pCO₂ may be sampled using the sensors at manually determined time points, or it may be automatically sampled (or both). In general, the controller(s) may continuously monitor and integrate any or all of these parameters.

In operation, the micro-bioreactor may be used for any appropriate types of cells, particularly for adherent and non-adherent mammalian cells, bacteria, and yeast. The micro-bioreactor may also be used to optimize processes such as cell growth and production of a desired product. Thus, them micro-bioreactor may be used to optimize conditions for growth and/or product production before scaling up (e.g., to 1000 L or more) production. This may be particularly useful in pharmaceutical (e.g., therapeutic antibody, etc.) production, or the production of other biologics or therapeutics. For example, in the production of cells or cell lines (e.g., standard cultures of difficult to grow cells, such as stem cells, non-transfected animal cells, etc.). Micro-bioreactors may also be useful in any process in which micro scale-up of a series of experiments or conditions is required for optimizing large-scale manufacturing. For example, micro-bioreactors may be useful in waste bioremediation studies for environmental clean-up and toxic waste abatement programs.

Unlike most previously available bioreactors, the micro-bioreactors described herein may be multi-well systems including small volume (“micro”) wells in a single plate (e., 6-well plate, etc.). The volume may be less than 1-3 ml/well on a single plate, which may have a single ABS format. Each well may be a fully integrated bioreactor, including biosensors for pH, pCO₂, pO₂, product formation, temperature, etc. Further, the cells may be non-invasively counted and imaged, and media flow into and out of each well may be individually controlled.

### Example 5

### Sensor Arrangement

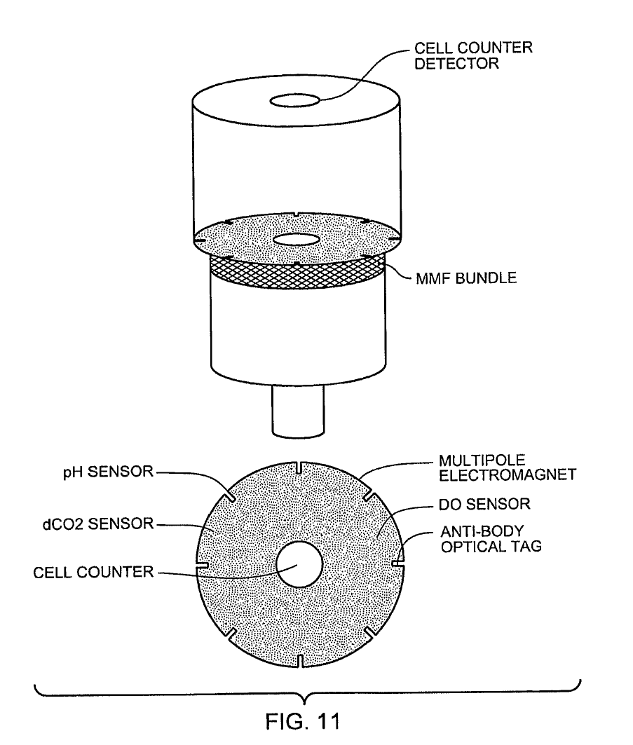

The sensors described herein may be arranged in any appropriate fashion. However, since each well may incorporate multiple non-invasive sensors (or combinations of sensors), the sensors may be arranged so that they do not interfere with each other. For example, the sensors may be arranged to avoid cross-talk (e.g., between different fluorescent sensors), or optical interference with other sensors or with cell counting or cell imaging. FIG. 11 shows one potentially advantageous arrangement of sensors, in which sensors for dissolved CO₂, dissolved O₂, pH, and product are arranged as concentrically around the perimeter of the well. The well is also shown as being surrounded by a multi-pole electromagnet that may be used in stirring the well.

In operation, a multimode fiber bundle with concentrically arranged fibers may be used with the dissolved O₂ (DO) sensor, dissolved CO2 (dCO2), ph sensor, and product-specific sensor (antibody-coated optical sensor). The concentrically arranged sensor patches permit attachment to the detectors/sensor telemetry (e.g., a fiber-optic cable bundle) without requiring alignment of the cable bundle. In one variation, the outer diameter of the cable bundle matches the outer diameter of the attachment to the well (e.g., the bottom of the well) for monitoring the sensors. In the arrangement of the sensors shown in FIG. 11, the center of the concentric sensor rings is left open, permitting unobstructed viewing (e.g., imaging) or cell counting. For example, a fiber-optic based turbidity-type cell counter may be used y centrally aligning an optical detector on top of each well, as shown in FIG. 11.

Using this concentric arrangement of sensors, optical signals may be simultaneously measured at user-specified (or automatic) intervals. Thus the sensors may simultaneously (or separately) measure any of: the level of product formation (or rate of product formation), cell counts, pH, DO, dCO₂, mixing rate, temperature, etc. Although FIG. 11 shows only a handful of sensors, additional sensors (or fewer sensors) may be used.

1. An environmentally isolated tissue culture device comprising:
an outer shell;
an inner shell enclosing at least one well, the well having an optically transparent base, wherein the transparent base is substantially thermally isolated from the outer shell by an air gap, and a long path of high thermal resistance between the base of the inner shell and the base of the outer shell;
a micro-heater configured to control the temperature of the one or more wells, the micro-heater comprising:
an optically transparent electrically conductive coating on the optically transparent base, and
one or more electrodes in electrical contact with the electrically conductive coating; and
a temperature sensor configured to provide feedback to control the micro-heater.

2. The tissue culture device of claim 1, wherein the temperature sensor is configured to detect the temperature of at least a portion of the optically transparent base.

3. The tissue culture device of claim 1, wherein the temperature sensor is mounted to a gimbaled mount.

4. The tissue culture device of claim 1, wherein the optically transparent electrically conductive coating comprises indium tin oxide (ITO).

5. The tissue culture device of claim 1, wherein the optically transparent electrically conductive coating is selected from the group consisting of: indium tin oxide (ITO), antimony tin oxide (ATO), fluorine-doped tin oxide (FTO), Cadmium oxide (CdO), or combinations thereof.

6. The tissue culture device of claim 1, further comprising an optically transparent protective layer over the optically transparent electrically conductive coating.

7. The tissue culture device of claim 6, wherein the optically transparent protective layer comprises SiO.

8. The tissue culture device of claim 1, wherein the inner shell comprises multiple wells.

9. The tissue culture device of claim 1, wherein the inner shell comprises six wells.

10. The tissue culture device of claim 1, wherein the optically transparent base comprises a glass substrate.

11. The tissue culture device of claim 1, wherein the optically transparent base is sufficiently thin to permit visualization by an inverted microscope.

12. The tissue culture device of claim 1, further comprising a lid.

13. The tissue culture device of claim 12, wherein the lid comprises a micro-heater configured to control the temperature of the lid.

14. The tissue culture device of claim 12, wherein the lid comprises an optically transparent surface configured to sit oppose the optically transparent base of the inner shell when the lid is engaged with the rest of the tissue culture device.

15. The tissue culture device of claim 14, wherein the optically transparent surface is temperature-controlled.

16. The tissue culture device of claim 12, further comprising a seal between the lid and the well.

17. The tissue culture device of claim 16, wherein the seal comprise an o-ring.

18. The tissue culture device of claim 1, further comprising a plurality of ports into each of the one or more wells.

19. The tissue culture device of claim 18, wherein at least some of the ports comprise a valve to regulate flow through the port.

20. The tissue culture device of claim 18, wherein at least some of the ports comprise septum ports.

21. The tissue culture device of claim 1, further comprising a port configured to connect to a supply of gas or gasses for cell culture.

22. The tissue culture device of claim 1, wherein the one or more electrodes comprise electrodes that are patterned into a tapered shape in contact with the electrically conductive coating.

23. The tissue culture device of claim 1, wherein the one or more electrodes comprise multiple pairs of electrodes of alternating or like polarity positioned about the periphery of the optically transparent base.

24. The tissue culture device of claim 23, wherein the multiple pairs of electrodes comprise at least two pairs of substantially “L-shaped” electrodes.

25. The tissue culture device of claim 1 further comprising a non-temperature sensor configured to detect a non-temperature parameter.

26. The tissue culture device of claim 25, wherein the non-temperature parameter is selected from the group consisting of: pH, dissolved O₂, dissolved CO₂, dissolved N₂, media level, product level, cell count, and combinations thereof.

27. The tissue culture device of claim 25, wherein the non-temperature sensor comprises an optical sensor.

28. The tissue culture device of claim 1, further comprising a controllable magnetic stirrer for stirring material within the well.

29. The tissue culture device of claim 28, wherein the controllable magnetic stirrer comprises a multi-pole magnetic source positioned around at least a portion of the outside of the well.

30. The tissue culture device of claim 29, wherein the multi-pole magnetic source comprises an electromagnet.

31. The tissue culture device of claim 28, further comprising beads configured to be acted on by the multi-pole magnetic source.

32. An environmentally isolated tissue culture device comprising:
an outer shell;
an inner shell enclosing at least one well, the well having an optically transparent base, wherein the inner shell is substantially thermally isolated from the outer shell;
a first micro-heater configured to control the temperature of the one or more wells;
a temperature sensor configured to provide feedback to control the first micro-heater; and
a sealable lid configured to seal over the well, wherein the lid comprises a second micro-heater configured to control the temperature of the lid.

33 The device of claim 32, wherein the micro-heater comprises an electrically conductive coating selected from the group consisting of: indium tin oxide (ITO), antimony tin oxide (ATO), fluorine-doped tin oxide (FTO), Cadmium oxide (CdO), or combinations thereof.

34. The device of claim 32, further comprising a second temperature sensor configured to sense the temperature of the sealable lid.

35. The device of claim 32, further comprising a controller configured to control the temperature of the first and second micro-heaters.

36. The device of claim 32, further comprising a plurality of ports accessing the well.

37. The device of claim 32, further comprising a gasket between the lid and the well.

38. The device of claim 32, further comprising a non-temperature sensor configured to sense a non-temperature parameter.

39. The device of claim 32, wherein the temperature of the lid is configured to be higher than the temperature of the well, thereby preventing substantial condensation on the lid.

40. An environmentally isolated tissue culture device comprising:
an outer shell;
an inner shell enclosing at least one well, the well having an optically transparent base, wherein the inner shell is substantially thermally isolated from the outer shell;
a micro-heater configured to control the temperature of the one or more wells,
a temperature sensor configured to provide feedback to control the micro-heater;
an integrated magnetic stirrer, wherein the integrated magnetic stirrer includes a multi-pole magnetic source positioned outside of the well; and
a lid configured to cover the well.

41. The device of claim 40, wherein the multi-pole magnetic source comprises an electromagnet.

42. The device of claim 40, further comprising one or more stirrers configured to be acted upon by the multi-pole magnetic source.

43. The device of claim 42, wherein the stirrers comprise magnetic or paramagnetic beads.

44. An environmental controller for controlling an environmentally isolated tissue culture device, comprising:
a pump configured to supply and/or withdraw material in a tissue culture device through a channel;
a valve regulating flow through the channel;
a temperature sensor input configured to receive information from a temperature sensor of the tissue culture device;
a temperature control output configured to apply power to a heater of the tissue culture device; and
a processing unit operably connected to the temperature sensor and the temperature control output, wherein the processing unit is configured to regulate the temperature of the tissue culture device;
a second sensor input configured to receive information from a non-temperature sensor of the tissue culture device, wherein the second sensor input is operably connected to the processing unit and the processing unit is further configured to regulate the non-temperature parameter sensed by the non-temperature sensor of the tissue culture device.

45. The controller of claim 44, wherein the non-temperature parameter is selected from the group consisting of: pH, dissolved O₂, dissolved CO₂, dissolved N₂, media level, product level, cell count, and combinations thereof.

46. The controller of claim 44, wherein the pump is configured to supply material selected from the group consisting of: media, O₂, CO₂, N₂, and gas mixtures thereof.

47 The controller of claim 44, further comprising an alarm coupled to the processing unit, wherein the alarm is selected from the group consisting of: audible, visible, tactile, and combinations thereof.

48. The controller of claim 47, wherein the alarm comprises a light.

49. The controller of claim 44, wherein the processing unit comprises a computer processor.

50. The controller of claim 44, wherein the processing unit is programmable.

51. The controller of claim 44, wherein the processing unit comprises a user interface.

52. The controller of claim 44, wherein the processing unit includes fluid control logic configured to manage the application and/or withdrawal of liquids and/or gasses in the tissue culture device.

53. The controller of claim 44, further comprising a plurality of sensor inputs configured to receive information from non-temperature sensors of the tissue culture device.

54. The controller of claim 44, wherein the temperature sensor input and the second sensor input comprise wireless receptors.

55. The controller of claim 44, further comprising a memory configured to store information from the temperature sensor and/or the non-temperature sensor.

56 The controller of claim 44, wherein the temperature control output comprises a cable configured to connect to electrodes of the tissue culture device.

57. The controller of claim 44, wherein the channel comprises tubing.

58. The controller of claim 44, where the pumps comprise fluid pumps

59. A system for regulating a tissue culture device comprising:
an environmentally isolated tissue culture device comprising:
an outer shell;
an inner shell enclosing at least one well, the well having an optically transparent base, wherein the inner shell is substantially thermally isolated from the outer shell;
a micro-heater configured to control the temperature of the well;
a temperature sensor configured sense the temperature of the well;
a plurality of ports accessing the well; and
a sealable lid configured to cover the well; and
a controller configured to control the environmentally isolated tissue culture device, the controller comprising:
a pump configured to supply and/or withdraw material in the well through a channel;
a valve regulating flow through the channel; and
a processing unit configured to receive information from the temperature sensor and operably connected to the micro-heater to regulate the temperature of the well, wherein the processing unit is configured to control the valve and pump to add or withdraw material in the well.

60. A method of regulating the micro-environment within a tissue culture device, wherein the tissue culture device includes at least one closed well having an optically transparent base coated with an optically transparent electrically conductive coating, the method comprising:
sensing the temperature of at least a portion of the well without opening the well;
applying power to at least one pair of electrodes in electrical contact with the electrically conductive coating to heat the base; and
sensing a non-temperature parameter from within the well without opening the well.

61. The method of claim 60 further comprising responding to the non-temperature parameter by adding or removing material from the well without opening the well.

62. The method of claim 60, wherein sensing the non-temperature parameter comprises sensing one or more of: pH, dissolved O₂, dissolved CO₂, dissolved N₂, media level, product level, cell count, and combinations thereof.
